SEQUENCE LISTING (5 sequences)

SEQ ID NO: 1 - protein, 630 aa, Homo sapiens
MELQPPEASIAVVSIPRQLPGSHSEAGVQGLSAGDDSELGSHCVAQTGLELLASGDPLPSASQNAEMIETGSDCVTQAGLQLLASSDPPALASKNAEVTGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 2 - protein, 529 aa, Homo sapiens
MSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 3 - protein, 568 aa, Homo sapiens
MELQPPEASIAVVSIPRQLPGSHSEAGVQGLSAGDDSGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 4 - protein, 661 aa, Homo sapiens
MELQPPEASIAVVSIPRQLPGSHSEAGVQGLSAGDDSELGSHCVAQTGLELLASGDPLPSASQNAEMIETGSDCVTQAGLQLLASSDPPALASKNAEVTETGFHHVSQADIEFLTSIDPTASASGSAGITGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 5 - protein, 631 aa, Homo sapiens
MELQPPEASIAVVSIPRQLPGSHSEAGVQGLSAGDDSETGSDCVTQAGLQLLASSDPPALASKNAEVTVETGFHHVSQADIEFLTSIDPTASASGSAGITGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

(19) United States
(12) Patent Application Publication
(10) Pub. No.: US 2025/0302987 A1
(43) Pub. Date: Oct. 2, 2025

(54) Targeted Lipid Nanoparticles

(71) Applicant: Zitra Medicines Pty Ltd, Carlton (AU)

(72) Inventor: Denzil Furtado, Carlton (AU)

(21) Appl. No.: 19/083,329

(22) Filed: Mar. 18, 2025

(30) Foreign Application Priority Data
Mar. 18, 2024 (AU) 2024900720

(51) Int. Cl. A61K 47/69; A61K 31/7105; A61K 47/68

(52) U.S. Cl. CPC A61K 47/6913; A61K 31/7105; A61K 47/6849

(57) ABSTRACT

The present invention relates to engineered targeted lipid nanoparticles (LNPs) comprising a nucleic acid, and compositions thereof, wherein the LNPs or compositions are capable of traversing the blood brain barrier (BBB) and delivering nucleic acid cargoes to a target tissue or cell in the central nervous system. In one aspect, the invention relates to the treatment of a neurological disease or disorder with a LNP or composition of the invention.

### FIELD OF THE INVENTION

The present invention relates to engineered targeted lipid nanoparticles (LNPs) comprising a nucleic acid, and compositions thereof, wherein the LNPs or compositions are capable of traversing the blood brain barrier (BBB) and delivering nucleic acid cargoes to a target tissue or cell in the central nervous system. In one aspect, the invention relates to the treatment of a neurological disease or disorder with a LNP or composition of the invention.

### INCORPORATION BY REFERENCE

The present application claims priority from Australian provisional application number 2024900720 filed on 18 Mar. 2024, the entire contents of which are incorporated herein by reference.

### REFERENCE TO A SEQUENCE LISTING

The application includes sequences in an electronic sequence listing named 627876SEQLST.XML of size 8.1 KB, created Jun. 24, 2025, which is incorporated by reference in its entirety.

### BACKGROUND OF THE INVENTION

Nucleic acid-based medicines such as messenger RNA (mRNA) hold promise to treat a vast array of diseases, but safe and effective delivery of the nucleic acids to disease-affected organs remains a major undertaking for the field. For example, effective non-invasive nucleic acid delivery for the central nervous system (CNS) is virtually non-existent, in large part due to the biological constraints imposed by the blood-brain barrier (BBB). The BBB is the most tightly regulated biological interface in the human body, separating the blood from the brain and carefully controlling the passage of molecules between the two compartments. The near impermeability of the BBB to most molecules is achieved by the concerted interaction of different brain cell types, including endothelial cells, vascular smooth muscle cells, pericytes, astrocytes, microglia and mast cells. This interaction produces a host of defense systems that are unique to the BBB, including a luminal glycocalyx, endothelial tight junctions and adherens junctions, and active efflux transporters. Thus, despite being well-perfused by an extensive cerebral vasculature, the brain is largely impenetrable to most exogenous molecules.

In light of the considerable biological challenge imposed by the BBB, existing methods to deliver large macromolecule drugs into the brain are mostly invasive. Treatments are often administered via surgical intervention in an inpatient setting, with the goal of bypassing the BBB altogether. Common routes of administration include intrathecal, intraparenchymal, and intraventricular infusion, the latter of which is commonly used for delivering gene therapies and recombinant protein drugs into the brain. Such invasive treatment procedures are suboptimal for several obvious reasons, including patient discomfort and the risks of brain tissue injury, infusion-site inflammation, and opportunistic infections.

Increasingly sophisticated studies of the BBB have uncovered mechanisms of biological transport to the brain that can be coopted using principled engineering strategies. Viral delivery systems, including adeno-associated viruses (AAVs), have been harnessed for CNS delivery via transport mechanisms that are still not well-understood however they are associated with potentially significant limitations such as toxicity and adverse immune responses.

In view of the above-described limitations and relative importance thereof of developing new strategies for the treatment of neurological diseases or disorders, there is a need for improved non-invasive therapeutics that overcome one or more of the above-described limitations.

Reference to any prior art in the specification is not an acknowledgement or suggestion that this prior art forms part of the common general knowledge in any jurisdiction or that this prior art could reasonably be expected to be combined with any other piece of prior art by a skilled person in the art.

### SUMMARY OF THE INVENTION

The inventor has developed a brain-targeted nucleic acid-LNP formulation conjugated with an antibody against the CD98hc protein, which is a subunit of the large neutral amino acid transporter (LAT1). The CD98hc-targeted compositions described herein exhibit robustly uniform pharmacokinetics across different nucleic acid cargoes, different CD98 antibody clones, and different LNP lipid structures. These findings therefore highlight that CD98-targeted mRNA-LNPs hold promise as a non-invasive therapeutic platform for the treatment of disparate neurological disorders. In particular, the technology described herein can be utilised as a “plug and play” platform that involves varying only the nucleic acid sequence to tailor the therapeutic for the treatment of specific neurological diseases and disorders. Advantageously, the versatility of this system allows for variation in the specific nucleic acid sequence, LNP composition, and CD98hc antibody clone, without compromising overall brain-targeting properties. The technology described herein is significant as it obviates the need for invasive surgical drug administration in the context of neurological diseases, instead allowing for intravenous dosing in an outpatient setting.

In an aspect of the invention, there is provided a liposome, lipoplex or a lipid nanoparticle (LNP) comprising a nucleic acid, wherein the liposome, lipoplex or LNP is conjugated to a non-ligand binder capable of transporting the liposome, lipoplex or LNP across the blood-brain barrier, wherein the non-ligand binder binds to CD98 heavy chain (CD98hc).

In another aspect, the present invention provides for a composition comprising a liposome, lipoplex or a lipid nanoparticle (LNP) and a nucleic acid, wherein the liposome, lipoplex or LNP is conjugated to a non-ligand binder capable of transporting the liposome, lipoplex or LNP across the blood-brain barrier, wherein the non-ligand binder binds to CD98 heavy chain (CD98hc).

In another aspect, there is provided a pharmaceutical composition comprising a liposome, lipoplex or a lipid nanoparticle (LNP) and a nucleic acid, wherein the liposome, lipoplex or LNP is conjugated to a non-ligand binder capable of transporting the liposome, lipoplex or LNP across the blood-brain barrier, wherein the non-ligand binder binds to CD98 heavy chain (CD98hc). In an embodiment, the pharmaceutical composition comprises a pharmaceutically acceptable carrier, diluent or excipient.

Accordingly, the present invention provides for a lipid nanoparticle (LNP) comprising a nucleic acid, wherein the LNP is conjugated to an antibody capable of transporting the LNP across the blood-brain barrier, wherein the antibody is an antibody against CD98 heavy chain (CD98hc), or a fragment thereof.

In another aspect, the present invention provides for a composition comprising a lipid nanoparticle (LNP) and a nucleic acid, wherein the LNP is conjugated to an antibody capable of transporting the LNP across the blood-brain barrier, wherein the antibody is an antibody against CD98 heavy chain (CD98hc), or a fragment thereof.

In another aspect, there is provided a pharmaceutical composition comprising a lipid nanoparticle (LNP) and a nucleic acid, wherein the LNP is conjugated to an antibody capable of transporting the LNP across the blood-brain barrier, wherein the antibody is an antibody against CD98 heavy chain (CD98hc) or a fragment thereof, and wherein the pharmaceutical composition comprises a pharmaceutically acceptable carrier, diluent or excipient.

In a preferred embodiment, the LNP encapsulates the nucleic acid. In another embodiment, the nucleic acid is bound to the LNP. In another embodiment, the nucleic acid is adsorbed on to the LNP.

In an embodiment, the LNP comprises an ionizable lipid, helper lipid, sterol, and PEG-lipid. In another embodiment, the LNP comprises an ionizable lipid, helper lipid, sterol, and a surfactant.

In an embodiment, the molar ratio of ionizable lipid, helper lipid, sterol, and PEG-lipid or surfactant, is 50:10:38.5:1.5. In an embodiment, the molar ratio of ionizable lipid, helper lipid, sterol, and PEG-lipid or surfactant is selected from the group consisting of 60:5:10:25, 55:30:45:0.2, 52:8:38.5:1.5, 52:8:37:3, 50:20:23.5:6.5 50:10.5:38:1.5, 50:12.5:35:2.5, 45:13:39.5:2.5, 35:16:46.5:2.5, 35:40:22.5:2.5, 26.5:20:52:1.5, 25:30:30:1, 40:10:38.5:1.5, 30:10:38.5:1.5, 40:10:38.5:1.5, 60:10:38.5:1.5, 70:10:38.5:1.5, 50:5:38.5:1.5, 50:15:38.5:1.5, 50:20:38.5:1.5, 50:25:38.5:1.5, 50:10:18.5:1.5, 50:10:28.5:1.5, 50:10:48.5:1.5, 50:10:58.5:1.5, 50:10:38.5:0.5, 50:10:38.5:1.0, 50:10:38.5:2.0, 50:10:38.5:2.5 or any combination thereof.

In an embodiment, the lipid nanoparticle comprises 20-60 mol % (e.g., 20-30 mol %, 20-40 mol %, 20-50 mol %, 20-60 mol %, 30-40 mol %, 30-50 mol %, 30-60 mol %, 40-50 mol %, 40-60 mol %, 45-55 mol %, or 45-50 mol % or 50-60 mol %) ionizable lipid; 5-25 mol % (e.g., 5-10 mol %, 5-15 mol %, 5-20 mol %, 5-25 mol %, or 10-15 mol %, 10-20 mol %, 10-25 mol %, 15-20 mol %, 15-25 mol %) helper lipid; 25-55 mol % (e.g., 25-35 mol %, 25-45 mol %, 25-55 mol %, 35-45 mol %, 35-55 mol % or 45-55 mol %) sterol; and 0.5-15 mol % (e.g., 0.5-5 mol %, 0.5-10 mol %, or 0.5-15 mol %, 2.5-5 mol %, 2.5-10 mol %, 2.5-15 mol %, 5-10 mol %, 5-15 mol %) PEG-lipid or surfactant.

In an embodiment, the ionizable lipid is selected from the group consisting of 9-Heptadecanyl 8-{(2-hydroxyethyl)[6-oxo-6-(undecyloxy)hexyl]amino}octanoate (SM-102), DLin-MC3-DMA (MC3), 1,2-distearyloxy-N,N-dimethyl-3-aminopropane (DSDMA), 1,2-dioleyloxy-N,Ndimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA), 2,5-bis((9z,12z)-octadeca-9,12,dien-1-yloxyl)benzyl-4-(dimethylamino)butnoate (LKY750), 4-hydroxybutyl)azanediyl)bis(hexane-6,1-diyl)bis(2-hexyldecanoate (ALC0315), C12-200, 306-012B, 4A3-SC8, cKK-E12, 2,2-dilinoleyl-4-dimethylaminoethyl-[1,3]-dioxolane (DLin-KC2-DMA), and di((Z)-non-2-en-1-yl) 9-((4-(dimethylamino)butanoyl)oxy)heptadecanedioate (L319). Preferably the ionizable lipid is SM-102 or MC3.

In an embodiment, the helper lipid is a cationic lipid. In this embodiment, the cationic lipid is selected from the group consisting of 1,2-dioleoyl-3-trimethylammonium-propane (DOTAP), 1,2-dioleoyl-s-glycero-3-ethylphosphocholine (EPC), dimethyldioctadecylammonium bromide (DDAB), and 1,2-di-O-octadecenyl-3-trimethylammonium-propane (DOTMA).

In an embodiment, the helper lipid is a charged lipid such as DOTAP or DOTMA.

In another embodiment, the helper lipid is a phospholipid. In this embodiment, the phospholipid is selected from the group consisting of 1,2-distearoyl-sn-glycero-3-phosphocholine (DSPC), 1,2-dioleoyl-sn-glycero-3-phosphoethanolamine (DOPE), 1,2-dioleoyl-3-trimethylammonium-propane (DOTAP), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), l-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine (DSPE), 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(l-glycerol) sodium salt (DOPG), and sphingomyelin and combinations thereof. Preferably, the phospholipid is DSPC or DOPE.

In an embodiment, the LNP comprises both a cationic lipid and a phospholipid described herein. In another embodiment, the LNP comprises a cationic lipid or a phospholipid described herein.

In an embodiment, the sterol is selected from the group consisting of β-sitosterol, cholesterol, 24(S)-hydroxycholesterol, 20α-hydroxycholesterol, cholesterol oleate, other cholesterol esters, fecosterol, campesterol, stigmasterol, brassicasterol, ergosterol, tomatidine, tomatine, ursolic acid and alpha-tocopherol and combinations thereof. Preferably, the sterol is β-sitosterol or cholesterol.

In another embodiment, the PEG-lipid is selected from the group consisting of PEG2000-c-DMG, PEG2000-DMG, PEG2000-DLPE, PEG2000-DMPE, PEG2000-DPPC, a PEG2000-DSPE lipid and combinations thereof. Preferably, the PEG2000-lipid is PEG2000-DSPE or PEG2000-DMG. In another embodiment, the PEG-lipid may be a different molecular weight as whose suitability is determined by a skilled person in the art. For example, the PEG-lipid may have a molecular weight of 200, 400, 600, 800, 1000, 1200, 1400, 1600, 1800, 2000, 2200, 2400, 2600, 2800, 3000, 3200, 3400, 3600 or 4000.

In an embodiment, the surfactant is selected from the group consisting of a non-ionic surfactant, an anionic surfactant, a cationic surfactant or an amphoteric surfactant. In an embodiment, the surfactant is Polysorbate 20 or Polysorbate 80.

In an embodiment, the LNP further comprises one or more additional helper lipids. For example, the LNP may comprise an additional permanently cationic or anionic lipid described herein or known in the art, or another lipid component such as but not limited to ceramide, sphingosine, sphingomyelin, cerebroside, LPC oleate, alpha-tocopherol, folate-conjugated lipid, dehydroascorbic acid-conjugated lipid or 6-O-glucose-conjugated lipid.

In a preferred embodiment, the ionizable lipid is SM-102, MC3 or ALC-0315; the helper lipid is a cationic lipid, preferably DOTAP, or a phospholipid, preferably DSPC or DOPE; the sterol is β-sitosterol or cholesterol and PEG-lipid is PEG2000-DSPE or PEG2000-DMG. In this embodiment, the molar ratio of ionizable lipid, helper lipid, preferably phospholipid, sterol, and PEG2000-lipid is 50:10:38.5:1.5.

In an embodiment, the composition further comprises a maleimide end-group modified PEG-lipid. Preferably, the maleimide end-group modified PEG-lipid is DSPE-PEG2000.

In an embodiment, the LNP further comprises a fluorescent label, chromophoric label, electron-dense label, chemiluminescent label or radioactive label.

In an embodiment, the CD98hc antibody is a human CD98hc antibody. In another embodiment, the CD98hc antibody is a mammalian CD98hc antibody.

In an embodiment, the CD98hc antibody targets human CD98hc having an amino acid sequence according to Uniprot IDs: P08195-1 (canonical isoform), P08195-2 (isoform 2), P08195-3 (isoform 3), P08195-4 (isoform 4), and P08195-5 (isoform 5).

In an embodiment, the CD98hc is glycosylated. In certain embodiments, the CD98hc is phosphorylated.

In an embodiment, the CD98hc antibody is an IgG antibody, optionally IgA1, IgA2, IgG1, IgG2, IgG3 and IgG.

In an embodiment, the CD98hc antibody is a full-length antibody. Alternatively, the CD98hc antibody is an antibody fragment selected from the group consisting of Fab, Fab′, Fab′-SH, F(ab′)2, Fv, single-domain (sdAb) and scFv. Preferably, the fragment is a sdAb (nanobody) or scFv.

In an embodiment, antibody binding to CD98hc does not inhibit amino acid transport by the CD98 heterodimeric complex. In another embodiment, antibody binding to CD98hc does not inhibit cell growth, cell adhesion, proliferation and/or apoptosis, mediated by the CD98 heterodimeric complex.

In an embodiment, the composition or LNP is suitable, or formulated for delivery to one or more or all tissues selected from the group consisting of the liver, spleen and/or lungs. In another embodiment, the composition or LNP is formulated for delivery across the blood-brain barrier.

In an embodiment, the composition further comprises a polymeric microparticle. In an embodiment, the mRNA is encapsulated in a polymeric microparticle. In an embodiment, the mRNA is bound to a polymeric microparticle. In another embodiment, the mRNA is adsorbed on to a polymeric microparticle.

In an embodiment, the composition further comprises an oil-in-water emulsion. For example, the mRNA is encapsulated in an oil-in-water emulsion. In an embodiment, the mRNA is bound to an oil-in-water emulsion. In another embodiment, the mRNA is adsorbed on to an oil-in-water emulsion. In another embodiment, the mRNA is resuspended in an oil-in-water emulsion.

In an embodiment, the nucleic acid is selected from the group consisting of a messenger RNA (mRNA) encoding a therapeutic or diagnostic polypeptide, deoxyribonucleic acid (DNA), small interfering RNA (siRNA), antisense oligonucleotide (ASO), small hairpin RNA (shRNA), micro-RNA (miRNA) or long non-coding RNA (lncRNA), preferably a mRNA.

In an embodiment, the mRNA comprises a cap, 5′UTR, coding sequence, a 3′UTR and a poly A tail.

In an embodiment, the mRNA comprises an optimised codon and/or a chemical modification. In one embodiment, the chemical modification is a uridine-5′-triphosphate nucleoside modification, optionally modified to N1-methylpseudouridine-5′-triphosphate (m¹ψTP) or 5-methoxyuridine-5′-triphosphate (5moUTP) and/or the optimised codon comprises substituting adenine (A) or uracil (U) containing codons with codons enriched in guanine (G) or cytosine (C). In an embodiment, the chemical modification increases mRNA stability and/or mRNA translation when compared to a mRNA without the chemical modification.

In an embodiment, the uridine modification is selected from the group consisting of pseudouridine (ψ), pyridin-4-one ribonucleoside, 5-aza-uridine, 6-aza-uridine, 2-thio-5-zauridine, 2-thio-uridine (s²U), 4-thio-uridine (s⁴U), 4-thiopseudouridine, 2-thio-pseudouridine, 5-hydroxy-uridine (ho5U), 5-aminoallyl-uridine, 5-halo-uridine (e.g., 5-iodo-uridine or 5-bromouridine), 3-methyluridine (m3U), 5-methoxy-uridine (5moU), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyluridine (chm⁵U), 5-carboxyhydroxymethyl-uridine methyl ester (mchm5U), 5-20 methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm5U), 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyluridine, 1-propynyl-pseudouridine, 5-taurinomethyl-uridine (tm⁵U), 1-taurinomethyl-pseudouridine, 5-taurinomethyl-2-thio-uridine (tm⁵s²U), 1-taurinomethyl-4-thio-pseudouridine, 5-methyl-uridine (m⁵U, i.e., having the nucleobase deoxythymine), 1-methylpseudouridine (m¹ψ), 5-methyl-2-thiouridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-3 0 thio-1-methyl-1-deaza-pseudouridine, dihydrouridine (D), dihydropseudouridine, 5,6-dihydrouridine, 5-methyldihydrouridine (m⁵D), 2-thio-dihydrouridine, 2-thiodihydropseudouridine, 2-methoxy-uridine, 2-methoxy-4-thio-uridine, 4-methoxy-pseudouridine, 4-methoxy-2-thio-pseudouridine, N1-methyl-pseudouridine (also known as 1-methylpseudouridine (m¹ψ), 3-(3-amino-3-carboxypropyl)uridine (acp³U), 1-methyl-3-(3-amino-3-carboxypropyl)pseudouridine (acp³ψ), 5-(isopentenylaminomethyl)uridine (inm⁵U), 5-(isopentenylaminomethyl)-2-thio-uridine (inm⁵s²U), a-thio-uridine, 2′-O-methyl-uridine (Um), 5,2′-O-dimethyl-uridine (m5Um), 2′-O-methyl-pseudouridine (Win), 2-thio-2′-O-methyl-uridine (s2Um), 5-methoxycarbonylmethyl-2′-O-methyl-uridine (mcm5Um), 5-carbamoylmethyl-2′-Omethyl-uridine (ncm5Um), 5-carboxymethylaminomethyl-2′-O-methyluridine (cmnm5Um), 3,2′O-dimethyl-uridine (m3Um), 5-(isopentenylaminomethyl)-2′-O-methyl-uridine (inm5Um), 1-thio-5 uridine, deoxythymidine, 2′-F-arauridine, 2′-F-uridine, 2′-OH-ara-uridine, 5-(2-carbomethoxyvinyl) uridine, and 5-[3-(1-E-propenylamino)]uridine. Preferably, the uridine modification is 5-methoxyuridine (5moU) or N1-methylpseudouridine (m¹ψ), more preferably m¹ψ.

In an embodiment, at least 10%, at least 20%, at least 30%, at least 40%, at least 45%, at least 50%, at least 55%, at least 60%, at least 65%, at least 70%, at least 75%, at least 80%, at least 85%, at least 90%, at least 95%, at least or 100% of uridines are modified.

In an embodiment, at least 10%, at least 20%, at least 30%, at least 40%, at least 45%, at least 50%, at least 55%, at least 60%, at least 65%, at least 70%, at least 75%, at least 80%, at least 85%, at least 90%, at least 95%, at least or 100% of uridines are modified to 5-methoxyuridine (5moU) or N1-methylpseudouridine (m¹ψ), preferably m¹ψ.

In an embodiment, each uridine-5′-triphosphate of a coding region of a mRNA of the invention is modified to 5-methoxyuridine-5′-triphosphate (5moUTP) or N1-methylpseudouridine-5′-triphosphate (m¹ψTP), preferably m¹ψTP.

In another embodiment, the nucleoside modification is a modified cytosine. In this embodiment, suitable cytidine modifications may be selected from the group consisting of 5-aza-cytidine, 6-aza-cytidine, pseudoisocytidine, 3-methyl-cytidine (m³C), N4-acetyl-cytidine (ac⁴C), 5-formyl-cytidine (PC), N4-methyl-cytidine (m⁴C), 5-methyl-cytidine (m⁵C), 5-methyl-cytidine 5′-triphosphate (5mCTP), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thiozebularine, 2-methoxy-cytidine, 2-methoxy-5-methyl-cytidine, 4-methoxy-pseudoisocytidine, 4-methoxy-1-methyl-pseudoisocytidine, lysidine (k²C), 2-thiocytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴₂Cm), 1-thiocytidine, 2′-F-ara-cytidine, 2′-F-cytidine, and 2′-OH-ara-cytidine.

In an embodiment, at least 10%, at least 20%, at least 30%, at least 40%, at least 45%, at least 50%, at least 55%, at least 60%, at least 65%, at least 70%, at least 75%, at least 80%, at least 85%, at least 90%, at least 95%, at least or 100% of cytosines are modified.

In an embodiment, at least 10%, at least 20%, at least 30%, at least 40%, at least 45%, at least 50%, at least 55%, at least 60%, at least 65%, at least 70%, at least 75%, at least 80%, at least 85%, at least 90%, at least 95%, at least or 100% of cytidine 5′-triphosphates are modified to 5-methylcytidine 5′-triphosphate (5mCTP).

In an embodiment, the optimised codon increases mRNA stability and/or mRNA translation when compared to a mRNA without the optimised codon. In another embodiment, the optimised codon increases guanine (G) and/or cytosine (C) codon content. Preferably, the optimised codon comprises substituting adenine (A) or uracil (U) containing codons with codons enriched in guanine (G) or cytosine (C).

In an embodiment, at least 10%, at least 20%, at least 30%, at least 40%, at least 45%, at least 50%, at least 55%, at least 60%, at least 65%, at least 70%, at least 75%, at least 80%, at least 85%, at least 90%, at least 95%, at least or 100% of codons are modified.

In an embodiment, at least 10%, at least 20%, at least 30%, at least 40%, at least 45%, at least 50%, at least 55%, at least 60%, at least 65%, at least 70%, at least 75%, at least 80%, at least 85%, at least 90%, at least 95%, at least or 100% of codons are modified to increase guanine (G) and/or cytosine (C) codon content.

In an embodiment, at least 10%, at least 20%, at least 30%, at least 40%, at least 45%, at least 50%, at least 55%, at least 60%, at least 65%, at least 70%, at least 75%, at least 80%, at least 85%, at least 90%, at least 95%, at least or 100% of adenine (A) or uracil (U) containing codons are substituted with codons enriched in guanine (G) or cytosine (C).

In an embodiment of the invention, at least 10%, at least 20%, at least 30%, at least 40%, at least 45%, at least 50%, at least 55%, at least 60%, at least 65%, at least 70%, at least 75%, at least 80%, at least 85%, at least 90%, at least 95%, at least or 100% of uridine of a coding region of a mRNA is modified to m¹ψ and at least 10%, at least 20%, at least 30%, at least 40%, at least 45%, at least 50%, at least 55%, at least 60%, at least 65%, at least 70%, at least 75%, at least 80%, at least 85%, at least 90%, at least 95%, at least or 100% of codons comprising A or U are substituted with codons enriched in guanine (G) or cytosine (C).

In a preferred embodiment of the invention, uridine of a coding region of a mRNA is modified to m¹ψ and codons comprising A or U are substituted with codons enriched in guanine (G) or cytosine (C).

In another aspect, there is provided a host cell, preferably a host cell of the central nervous system, liver, spleen or lungs, comprising a nucleic acid of the invention. In an embodiment, the host cell is a hepatocyte, reticular cell, lymphocyte, alveolar epithelial cell, a glial cell including astrocytes, oligodendrocytes, ependymal cells, and microglia, or a neuronal cell. In an embodiment, the host cell is obtained from a subject having a neurological disease or disorder described herein. In an embodiment, the host cell is a human or mammalian cell.

In another aspect, there is provided a method of synthesis of a LNP described herein, the method comprising:
(i) generating a lipid nanoparticle comprising a nucleic acid; and
(ii) conjugating the LNP with an antibody against CD98 heavy chain (CD98hc),
wherein the antibody is capable of transporting the LNP across the blood-brain barrier.

In an embodiment, the LNP is conjugated with an antibody against CD98hc via a thiol-maleimide reaction. In a preferred embodiment, conjugating the LNP with an antibody against CD98hc comprises post-inserting a maleimide end-group modified PEG-lipid into the LNP, and conducting a thiol-maleimide reaction with the antibody, wherein the antibody has been labeled with N-succinimidyl S-acetylthioacetate (SATA). In an embodiment, the maleimide end-group modified PEG-lipid is a DSPE-PEG lipid.

In an embodiment, the LNP comprises an ionizable lipid, a helper lipid such as a phospholipid or cationic lipid, a sterol, and a PEG-lipid or surfactant described herein. In an embodiment, the molar ratio of ionizable lipid, phospholipid, sterol, and PEG-lipid or surfactant is any of the ratios or mol % values described herein. In a preferred embodiment, the ionizable lipid is SM-102, MC3, or ALC-0315, the phospholipid is DSPC, DOTAP or DOPE, the sterol is β-sitosterol or cholesterol and the PEG-lipid is PEG-DSPE or PEG-DMG. In this embodiment, the nucleic acid is preferably mRNA. In this embodiment, the LNP further comprises a maleimide end-group modified PEG-lipid. In an embodiment, the LNP further comprises a maleimide end-group modified DSPE-PEG lipid.

In an embodiment, the method further comprises producing a therapeutic or diagnostic nucleic acid, preferably mRNA, from a DNA polynucleotide, and encapsulating the nucleic acid within the LNP. In an embodiment, the method further comprises labelling the LNP with a lipid fluorescent label.

Preferably, the method of producing the nucleic acid is carried out using a cell free assay, preferably in vitro transcription. In an embodiment, the method further comprises introducing a chemical modification to the mRNA, preferably a uridine nucleoside modification.

In an embodiment, the DNA polynucleotide is provided in a plasmid or vector.

In an aspect the present invention provides a LNP obtained from a method described herein.

In another aspect, there is provided a kit comprising a LNP of the invention, or a composition of the invention.

In another aspect, there is provided a method of treating or preventing a neurological disease or disorder in a subject in need thereof, comprising administering a LNP or pharmaceutical composition of the invention to the subject, thereby treating or preventing the neurological disease or disorder in the subject.

In another aspect, there is provided use of a LNP or pharmaceutical composition of the invention in the manufacture of a medicament for treating or preventing a neurological disease or disorder in a subject.

In another aspect, there is provided a LNP or pharmaceutical composition of the invention, for use in treating or preventing a neurological disease or disorder in a subject in need thereof.

In an embodiment, the method further comprises identifying a subject having, or at risk of having a neurological disease or disorder.

In an embodiment, the subject exhibits one or more of the following symptoms: difficulty coordinating movement (ataxia), abnormal eye movements (vertical supranuclear gaze palsy), poor muscle tone (hypotonia), difficulty with speech, difficulty with swallowing and feeding, loss of cognitive skills, seizures.

In an embodiment, the treatment improves one or more of the above symptoms by at least 10%, at least 20%, at least 30%, at least 40%, at least 45%, at least 50%, at least 55%, at least 60%, at least 65%, at least 70%, at least 75%, at least 80%, at least 85%, at least 90%, at least 95%, or more when compared to a subject not having a neurological disease or disorder.

In an embodiment, where prevention of a neurological disease or disorder is contemplated, the subject preferably has no clinical symptoms of the neurological disease or disorder.

In an embodiment, the treatment further comprises administering an additional therapeutic agent or compound. In an embodiment, the additional therapeutic agent or compound is bound to the LNP. In an embodiment, the LNP encapsulates the additional therapeutic agent or compound. In an embodiment, the additional therapeutic agent or compound is adsorbed on to the LNP.

In this embodiment, administration of the additional therapeutic may be at the same time or a different time to the administration of a composition of the invention.

In an embodiment, the nucleic acid is expressed in cells of the central nervous system including one or more or all of neurons, astrocytes, ependymal cells, glial cells and oligodendrocytes.

In another aspect, there is provided a method of expressing a nucleic acid in a cell of the central nervous system of a subject, comprising administering a LNP or composition of the invention to the subject, thus expressing the nucleic acid in the cell of the subject.

In another aspect, there is provided use of a LNP or composition of the invention in the manufacture of a medicament for expressing a nucleic acid in a cell of the central nervous system of a subject.

In another aspect, there is provided a LNP or composition of the invention for use in expressing a nucleic acid in a cell of the central nervous system of a subject.

Preferably, the nucleic acid is a mRNA and the cell is selected from the group consisting of glial cells including astrocytes, oligodendrocytes, ependymal cells and microglia, and a neuronal cell.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or group of compositions of matter.

The invention is hereinafter described by way of the following non-limiting Examples and with reference to the accompanying FIGS.

### DESCRIPTION OF THE DRAWINGS

FIG. 1. Engineered CD98hc-targeted LNPs. Mechanism of transport of antibody conjugated mRNA encapsulated LNPs targeted against CD98hc through the BBB into the brain parenchyma.

FIG. 2A. Experimental procedure for in vivo administration and assessment of CD98hc-targeted mRNA-LNPs. FIG. 2B. FLuc mRNA expression and nanoparticle biodistribution (DiD) at 24 h post-administration, visualised via ex vivo whole organ fluorescence imaging. FIG. 2C. Expression of mFlame mRNA at 24 h post-administration, visualised via ex vivo whole organ fluorescence imaging. FIGS. 2D-E. Confocal microscopy of (D) mFlame and (D)NGFR expression in the brain 24 h post-administration of CD98hc-targeted mRNA-LNP.

FIGS. 3A-B. mRNA expression (Fluc) and nanoparticle biodistribution (DiD) profile in peripheral organs 24 h after intravenous administration of CD98hc-targeted mRNA-LNPs. FIG. 3C. Fluc mRNA expression profile in inguinal, axial and cervical lymph nodes. FIG. 3D. Net charge of CD98hc-targeted and control mRNA-LNP formulations.

FIGS. 4A-C. Brain mRNA expression profile 24 h after intravenous administration of CD98hc-targeted mRNA-LNPs functionalised with three different CD98hc antibody clones (A-C respectively). FIGS. 4D-G. Brain mRNA expression and nanoparticle biodistribution (DiD) profile 24 h after intravenous administration of CD98hc-targeted mRNA-LNPs comprising different ionizable lipids (D-E), sterols (F), and phospholipids (G).

FIG. 5A: Schematic depicting intravenous administration of CD98hc-tLNPs encapsulating Cre mRNA into Ai14 mice, followed by visualization of tdTomato expression and LNP biodistribution (via DiR lipid dye signal) in the brain 3 days after administration.

FIG. 5B: CD98hc-tLNPs encapsulating Cre mRNA and formulated with DSPE-PEG2000 instead of DMG-PEG2000 produce higher tdTomato expression in the brains of Ai14 mice 3 days after intravenous administration.

FIG. 5C: Quantitation of tdTomato expression in whole brains (top graph) and sagittal hemispheres (bottom graph) of mice 3 days after intravenous administration of PBS, CD98hc-tLNPs formulated with DSPE-PEG2000, or CD98hc-tLNPs formulated with DMG-PEG2000.

FIG. 5D: Visualization (top) and quantitation (bottom) of LNP localization in the brains of Ai14 mice via DiR dye signal 3 days after intravenous administration of PBS, CD98hc-tLNPs formulated with DSPE-PEG2000, or CD98hc-tLNPs formulated with DMG-PEG2000.

FIGS. 6A-B. CD98hc-tLNPs are compatible with different ionizable lipids (A) SM102 and (B) 4A3-Sc8, and retain efficient brain-targeting independent of specific ionizable lipid chemistries. tdTomato expression in Ai14 mouse brain 3 days after IV injection of CD98hc-tLNPs formulated with Cre mRNA.

FIG. 7. CD98hc-tLNPs show brain-wide BBB engagement and mRNA expression after IV injection. tdTomato/DAPI expression in Ai14 mice 3 days after Cre mRNA administration.

FIG. 8A: Organ LNP biodistribution profile of CD98hc-tLNPs 24 h after intravenous administration, as measured by DiR dye signal.

FIG. 8B: CD98hc-tLNPs have significantly reduced mRNA expression (as measured by FLuc bioluminescence) and LNP localization (as measured by DiR dye signal) in the liver compared to untargeted LNPs 24 h after intravenous administration.

### DETAILED DESCRIPTION OF THE EMBODIMENTS

Messenger RNA (mRNA) medicines hold promise to treat a vast array of diseases, but safe and effective mRNA delivery to disease-affected organs remains a major undertaking for the field. In particular, effective non-invasive mRNA delivery systems for the treatment of neurological disorders or diseases prevalent in the central nervous system (CNS) are virtually non-existent, in large part due to the biological constraints imposed by the blood-brain barrier (BBB). Whilst viral delivery systems, including adeno-associated viruses (AAVs), have been harnessed for CNS delivery, such approaches are associated with potentially significant side effects including toxicity and adverse immune responses. Recognizing the many drawbacks of viral delivery, the inventor sought to develop a non-viral delivery system capable of producing functional nucleic acid expression in the brain in vivo after intravascular administration.

In particular, the inventor has developed a brain-targeted nucleic acid-LNP formulation conjugated with antibodies against the CD98 heavy chain protein (CD98hc; also known 4F2), which is the heavy chain subunit of the large neutral amino acid transporter (LAT1). CD98hc is a BBB transport protein however, prior to this current work it remained unclear whether CD98hc-targeted antibodies could also facilitate the delivery of much larger, conjugated cargoes into the brain.

Herein, the inventor has engineered targeted nucleic acid-encapsulated lipid nanoparticles (LNPs) bearing monoclonal antibodies against the CD98hc transporter. Intravenous administration of these CD98hc-targeted nucleic acid-LNPs produces functional mRNA expression in the brain, as well as in peripheral secondary lymphoid tissues. Localization of these formulations in the CNS is a distinct property of CD98hc binding, as untargeted nucleic acid-LNPs and nucleic acid-LNPs targeted against a different antigen fail to produce any brain-specific mRNA expression. CD98hc-targeted formulations exhibit robustly uniform pharmacokinetics across different nucleic acid cargoes, different CD98hc antibody clones, and different LNP lipid structures. These findings therefore suggest that CD98hc-targeted mRNA-LNPs may hold promise as a non-invasive therapeutic platform for the treatment of disparate CNS disorders whereby the described technology can be utilised as a “plug and play” platform that involves varying only the nucleic acid sequence to tailor the therapeutic for the treatment of specific CNS diseases and disorders. Such an approach is promising in that it obviates the need for invasive surgical drug administration in the context of brain diseases, instead allowing for intravenous dosing in an outpatient setting. Significantly, the versatility of this system also allows for variation in the specific mRNA sequence, LNP composition, and CD98hc antibody clone, without compromising overall brain-targeting properties.

### General Techniques and Definitions

Unless specifically defined otherwise, all technical and scientific terms used herein shall be taken to have the same meaning as commonly understood by one of ordinary skill in the art (e.g., in cell culture, molecular genetics, molecular biology, protein chemistry and biochemistry).

Unless otherwise indicated, the recombinant polynucleotide, polypeptide, cell culture, and immunological techniques utilized in the present invention are standard procedures, well known to those skilled in the art. Such techniques are described and explained throughout the literature in sources such as Perbal (1984), Sambrook (1989), Brown (1991), Glover and Hames (1995 and 1996), Ausubel et al. (1988) and Coligan et al. (including all updates until present).

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10%, more preferably 5%, more preferably 1%, of the particular term.

The term “antibody” herein is used in the broadest sense and specifically includes full length monoclonal antibodies, polyclonal antibodies, and, unless otherwise stated or contradicted by context, antigen-binding fragments, antibody variants, so long as they exhibit the desired biological activity. Generally, a full-length antibody is a glycoprotein comprising at least two heavy (H) chains and two light (L) chains inter-connected by disulfide bonds, or an antigen binding portion thereof. Each heavy chain is comprised of a heavy chain variable region (abbreviated as VH) and a heavy chain constant region. The heavy chain constant region is comprised of three domains, CHI, CH2 and CH3. Each light chain is comprised of a light chain variable region (abbreviated as VL) and a light chain constant region. The light chain constant region is comprised of one domain, CL. The VH and VL regions can be further subdivided into regions of hypervariability, termed complementarily determining regions (CDR), interspersed with regions that are more conserved, termed framework regions (FR). Each VH and VL is composed of three CDRs and four FRs, arranged from amino-terminus to carboxy-terminus in the following order: FRI, CDRI, FR2, CDR2, FR3, CDR3, FR4. The variable regions of the heavy and light chains contain a binding domain that interacts with an antigen. General principles of antibody molecule structure and various techniques relevant to the production of antibodies are provided in, e.g., Harlow and Lane, Antibodies: A Laboratory Manual, Cold Spring Harbor Laboratory Press, Cold Spring Harbor, N. Y., (1988).

Depending on the amino acid sequence of the constant domain of their heavy chains, full length antibodies can be assigned to different “classes”. There are five major classes of full length antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known.

The term “monoclonal antibody” as used herein refers to an antibody obtained from a population of substantially homogeneous antibodies, i.e., the individual antibodies comprising the population are identical and/or bind the same epitope, except for possible variants, e.g., containing naturally occurring mutations or that may arise during production of the monoclonal antibody, such variants generally being present in minor amounts. In contrast to polyclonal antibody preparations, which typically include different antibodies directed against different determinants (epitopes), each monoclonal antibody of a monoclonal antibody preparation is directed against a single determinant on the antigen. The term “monoclonal” indicates the character of the antibody as being obtained from substantially homogeneous population of antibodies, and is not to be construed as requiring production of the antibody by any particular method. For example, the monoclonal antibodies to be used in accordance with the present invention may be made by a variety of techniques, including but not limited to the hybridoma method (see, e.g., Kohler et al., Nature, 256:495 (1975)), recombinant DNA methods (see, e.g., U.S. Pat. No. 4,816,567), phage-display methods (e.g., using the techniques described in Clackson et al., Nature, 352:624-628 (1991) and Marks et al., J Mal. Biol., 222:581-597 (1991)), and methods utilizing transgenic animals containing all or part of the human immunoglobulin loci, such methods and other exemplary methods for making monoclonal antibodies being described herein. Specific examples of monoclonal antibodies herein include chimeric antibodies, humanized antibodies, and human antibodies, including antigen-binding fragments thereof.

The term “recombinant antibody”, as used herein, refers to an antibody (e.g. a chimeric, humanized, or human antibody or antigen-binding fragment thereof) that is expressed by a recombinant host cell comprising nucleic acid encoding the antibody. Examples of “host cells” for producing recombinant antibodies include: (1) mammalian cells, for example, Chinese Hamster Ovary (CHO), COS, myeloma cells (including YO and NSO cells), baby hamster kidney (BHK), Hela and Vero cells; (2) insect cells, for example, sf9, sf21 and Tn5; (3) plant cells, for example plants belonging to the genus Nicotiana (e.g. Nicotiana tabacum); (4) yeast cells, for example, those belonging to the genus Saccharomyces (e.g. Saccharomyces cerevisiae) or the genus Aspergillus (e.g. Aspergillus niger); (5) bacterial cells, for example Escherichia coli cells or Bacillus subtilis cells, etc.

An “antigen-binding fragment” of an antibody is a molecule that comprises a portion of a full-length antibody which is capable of detectably binding to the antigen, typically comprising one or more portions of at least the VH region. Antigen-binding fragments may include single-chain constructs wherein the VL and VH regions, or selected portions thereof, are joined by synthetic linkers or by recombinant methods to form a functional, antigen-binding molecule.

Antigen-binding fragments can also be a single-domain antibody (sdAb), also known as a nanobody, which is an antibody fragment consisting of a single monomeric variable antibody domain (VHH). While some antigen-binding fragments of an antibody can be obtained by actual fragmentation of a larger antibody molecule (e.g., enzymatic cleavage), most are typically produced by recombinant techniques. Such antibodies can be prepared as full-length antibodies or antigen-binding fragments thereof. Examples of antigen-binding fragments include Fab, Fab′, F(ab)2, F(ab1)2, F(ab)3, Fv (typically the VL and VH domains of a single arm of an antibody), single-chain Fv (scFv, see e.g., Bird et al., Science 1988; 242:423-426; and Huston et al. PNAS 1988; 85:5879-5883), dsFv, Fd (typically the VH and CHI domain), and dAb (typically a VH domain) fragments; VH, VL, VHH, and V-NAR domains; monovalent molecules comprising a single VH and a single VL chain; minibodies, diabodies, triabodies, tetrabodies, and kappa bodies (see, e.g., Ill et al., Protein Eng 1997; 10:949-57); camel IgG; IgNAR; as well as one or more isolated CDRs or a functional paratope, where the isolated CDRs or antigen-binding residues or polypeptides can be associated or linked together so as to form a functional antibody fragment. Various types of antibody fragments have been described or reviewed in, e.g., Holliger and Hudson, Nat Biotechnol 2005; 23:1126-1136; WO2005040219, and published U.S. Patent Applications 20050238646 and 20020161201. Antibody fragments can be obtained using conventional recombinant or protein engineering techniques, and the fragments can be screened for antigen-binding or other function in the same manner as are intact antibodies.

Various techniques have been developed for the production of antibody fragments. Traditionally, these fragments were derived via proteolytic digestion of full-length antibodies (see, e.g., Morimoto et al., Journal of Biochemical and Biophysical Methods, 24:107-117 (1992); and Brennan et al., Science, 229:81 (1985)). However, these fragments can now be produced directly by recombinant host cells. Alternatively, Fab′-S fragments can be directly recovered from E. coli and chemically coupled to form F(ab′)2 fragments (Carter et al., Bio/Technology, 10:163-167 (1992)). According to another approach, F(ab′)2 fragments can be isolated directly from recombinant host cell culture. In other embodiments, the antibody of choice is a single chain Fv fragment (scFv). See WO 1993/16185; U.S. Pat. Nos. 5,571,894; and 5,587,458. The antibody fragment may also be a “linear antibody”, e.g., as described in U.S. Pat. No. 5,641,870, for example.

The term “antibody derivative” as used herein refers to a molecule comprising a full-length antibody or an antigen-binding fragment thereof, wherein one or more amino acids are chemically modified or substituted. Chemical modifications that can be used in antibody derivative includes, e.g., alkylation, PEGylation, acylation, ester formation or amide formation or the like, e.g., for linking the antibody to a second molecule. Exemplary modifications include PEGylation (e.g., cysteine-PEGylation), biotinylation, radiolabeling, and conjugation with a second agent (such as a cytotoxic agent).

The term “human antibody” as used herein, is intended to include antibodies having variable regions in which both the framework and CDR regions are derived from (i.e., are identical or essentially identical to) human germline immunoglobulin sequences. Furthermore, if the antibody contains a constant region, the constant region also is “derived from” human germline immunoglobulin sequences. The human antibodies of the invention can include amino acid residues not encoded by human germline immunoglobulin sequences (e.g., mutations introduced by random or site-specific mutagenesis in vitro or by somatic mutation in viva). However, the term “human antibody”, as used herein, is not intended to include antibodies in which CDR sequences derived from the germline of another mammalian species, such as a mouse, have been grafted onto human framework sequences.

A “humanized” antibody is a human/non-human chimeric antibody that contains a minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit, or non-human primate having the desired specificity, affinity, and capacity. In some instances, FR residues of the human immunoglobulin are replaced by corresponding non-human residues. Furthermore, humanized antibodies can comprise residues that are not found in the recipient antibody or in the donor antibody. These modifications are made to further refine antibody performance. In general, a humanized antibody will comprise substantially all of at least one, and typically two, variable domains, in which all or substantially all of the hypervariable loops correspond to those of a nonhuman immunoglobulin and all or substantially all of the FR residues are those of a human immunoglobulin sequence. The humanized antibody can optionally also comprise at least a portion of an immunoglobulin constant region (Fe), typically that of a human immunoglobulin.

The term “hypervariable region” refers to the amino acid residues of an antibody that are responsible for antigen binding. The hypervariable region generally comprises amino acid residues from a “complementarity-determining region” or “CDR” (residues 24-34 (LI), 50-56 (L2) and 89-97 (L3) in the light-chain variable domain and 31-35 (HI), 50-65 (H2) and 95-102 (H3) in the heavy-chain variable domain; (Kabat et al. (1991) Sequences of Proteins of Immunological Interest, Fifth Edition, U.S. Department of Health and Human Services, NIH Publication No. 91-3242) and/or those residues from a “hypervariable loop” (residues 26-32 (LI), 50-52 (L2) and 91-96 (L3) in the light-chain variable domain and 26-32 (HI), 53-55 (H2) and 96-101 (H3) in the heavy-chain variable domain; Chothia and Lesk, J. Mol. Biol. 1987; 196:901-917). Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide can contain fewer or additional amino acids corresponding to a shortening of, or insertion into, a FR or CDR of the variable domain. For example, a heavy chain variable domain can include a single amino acid insert (residue 52a according to Kabat) after residue 52 of CDR H2 and inserted residues (e.g. residues 82a, 82b, and 82c, etc. according to Kabat) after heavy chain FR residue 82. The Kabat numbering of residues can be determined for a given antibody by alignment at regions of homology of the sequence of the antibody with a “standard” Kabat numbered sequence.

“Framework region” or “FR” residues are those VH or VL residues other than the CDRs as herein defined.

An “epitope” or “binding site” is an area or region on an antigen to which an antigen binding peptide (such as an antibody) specifically binds. A protein epitope can comprise amino acid residues directly involved in the binding (also called the immunodominant component of the epitope) and other amino acid residues, which are not directly involved in the binding, such as amino acid residues which are effectively blocked by the specifically antigen binding peptide (in other words, the amino acid residue is within the “solvent-excluded surface” and/or “footprint” of the specifically antigen binding peptide).

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For a review of methods for assessment of antibody purity, see, e.g., Flatman et al, J. Chromatogr. B 848:79-87 (2007).

“Specific binding” or “specifically binds” or “binds” refer to antibody binding to an antigen or an epitope within the antigen with greater affinity than for other antigens. Ko is the equilibrium dissociation constant between the antibody and its antigen. Ko and affinity are inversely related. The “on-rate” (kon) is a constant used to characterize how quickly the antibody binds to its target. The “off-rate” (koff) is a constant used to characterize how quickly an antibody dissociates from its target. The dissociation constant Ko can be measured using standard procedures. For example, the Ko of an antibody can be determined by using surface plasmon resonance, such as by using a biosensor system, e.g., a Biacore® system, or by using bio-layer interferometry technology. The smaller the value of the Ko of an antibody, the higher affinity that the antibody binds to a target antigen. Antibodies that specifically bind to the antigen or the epitope within the antigen can, however, have cross-reactivity to other related antigens, for example to the same antigen from other species (homo logs), such as human or monkey, for example Macaca fascicularis (cynomolgus, cyno), Pan troglodytes (chimpanzee, chimp) or Callithrix jacchus (common marmoset, marmoset).

The blood-brain barrier (BBB) refers a physiological barrier between the peripheral circulation and the brain and spinal cord which is formed by tight junctions within the brain capillary endothelial plasma membranes, creating a tight barrier that restricts the transport of molecules into the brain. The BBB can restrict the transport of even very small molecules such as urea (60 Daltons) into the brain. Examples of the BBB include the BBB within the brain, the blood-spinal cord barrier within the spinal cord, and the blood-retinal barrier within the retina, all of which are contiguous capillary barriers within the CNS. The BBB also encompasses the blood-CSF barrier (choroid plexus) where the barrier is comprised of ependymal cells rather than capillary endothelial cells.

A blood-brain barrier receptor is an extracellular membrane-linked receptor protein expressed on brain endothelial cells which is capable of transporting molecules across the BBB or be used to transport exogenous administrated molecules. Examples of blood-brain barrier receptors include, but are not limited to, Large neutral Amino acid Transporter (LAT) complex, including CD98 component, transferrin receptor (TfR), insulin receptor, insulin-like growth factor receptor (IGF-R), low density lipoprotein receptors including without limitation low density lipoprotein receptor-related protein I (LRPI) and low density lipoprotein receptor-related protein 8 (LRP8), and heparin-binding epidermal growth factor-like growth factor (HB-EGF). An exemplary blood-brain barrier receptor herein is CD98hc.

The central nervous system (CNS) refers to the complex of nerve tissues that control bodily function and includes the brain and spinal cord.

A conjugate as used herein refer to a protein covalently linked to one or more heterologous molecule(s), including but not limited to a therapeutic peptide or protein, an antibody, a label, or a neurological disorder drug.

As used herein the term coupled refers to the joining or connection of two or more objects together. When referring to chemical or biological compounds, coupled can refer to a covalent connection between the two or more chemical or biological compounds. By way of a non-limiting example, an antibody of the invention can be coupled with a peptide of interest to form an antibody coupled peptide. An antibody coupled peptide can be formed through specific chemical reactions designed to conjugate the antibody to the peptide. In certain embodiments, an antibody of the invention can be covalently coupled with a peptide of the invention through a linker. The linker can, for example, be first covalently connected to the antibody or the peptide, then covalently connected to the peptide or the antibody.

An effective amount or therapeutically effective amount of an agent, e.g., a pharmaceutical formulation, refers to an amount effective, at dosages and for periods of time necessary, to achieve the desired therapeutic or prophylactic result.

A linker refers to a chemical linker or a single chain peptide linker that covalently connects two different entities. A linker can be used to connect any two of an antibody or a fragment thereof, a blood brain barrier shuttle, a fusion protein and a conjugate of the present invention. The linker can connect, for example, the VH and VL in scFv, or the monoclonal antibody or antigen-binding fragment thereof with a therapeutic molecule, such as a second antibody. Single chain peptide linkers, comprised of from 1 to 25 amino acids, such as 1, 2, 3, 4, 5, 6, 7, 8, 9, 10, 11, 12, 13, 14, 15, 16, 17, 18, 19, 20, 21, 22, 23, 24, or 25 amino acids, joined by peptide bonds, can be used. In certain embodiments, the amino acids are selected from the twenty naturally occurring amino acids. In other embodiments, one or more of the amino acids are selected from glycine, alanine, proline, asparagine, glutamine and lysine. Chemical linkers, such as a hydrocarbon linker, a polyethylene glycol (PEG) linker, a polypropylene glycol (PPG) linker, a polysaccharide linker, a polyester linker, a hybrid linker consisting of PEG and an embedded heterocycle, and a hydrocarbon chain can also be used.

### Cluster of Differentiation 98 Heavy Chain Subunit

Cluster of differentiation 98 heavy chain subunit (CD98hc) is a member of the solute carrier family and heterodimerizes with a number of CD98 light chain members to form amino acid transporters at the BBB (Zuchero Y J, et al. Neuron. 2016; 89(1): 70-82, citing Boado R J, et al. PNAS 1999; 96(21): 12079-84). The intracellular portion of CD98hc functions to mediate integrin signaling, which plays a role in both cell growth and tumorigenesis (Zuchero Y J, et al. Neuron. 2016; 89(1): 70-82, Cantor J M and Ginsburg M H. J Cell Sci 2012; 125: 1373-82). CD98hc is highly expressed on human brain microvasculature.

The term CD98hc as used herein, refers to an integral membrane protein consisting of a CD98hc that links to any of multiple light chains by a disulfide bond. When associated with LAT1 or LAT2, the heterodimer transporter complexes behave as obligatory amino acid exchangers. Human CD98hc is encoded by the SLC3 A2 gene.

CD98hc as used herein refers to any native CD98hc from any vertebrate source, including mammals such as primates (e.g., humans) and rodents (e.g., mice and rats), unless otherwise indicated. CD98hc is also known by the names, inter alia, SLC3A2, 4F2, 4F2hc, Mdul, Ly 10, Mdvl, Frpl, Mgp2, Mgp2hc, NACAE, 4T2, 4T2hc, and TROP4. The term CD98hc encompasses “full-length,” unprocessed CD98hc as well as any form of CD98hc which results from processing in the cell. The term also encompasses naturally occurring variants of CD98hc, e.g., splice variants or allelic variants. CD98hc is 80 kDa type II transmembrane protein and pairs with 6 different light chain members or “binding partners” of the L-type amino transporter family of about 40 kDa (LAT1, LAT2, y+LAT1, y+LAT2, xCT, Ase) by a disulfide bond to form a heterodimeric complex. Thus, as used herein, “CD98 heterodimeric complex” refers to protein complexes comprising the CD98 heavy chain (e.g., LAT1/CD98hc, LAT2/CD98hc, y+LAT1/CD98hc, y+LAT2/CD98hc, xCT/CD98hc, and/or Asc/CD98hc). The CD98 heterodimeric complex functions as an amino acid transporter.

Functionally, CD98hc is required for the surface expression and basolateral localization of the amino acid transporter complex in polarized epithelial cells. CD98hc also interacts with beta 1 integrins and regulates their activation through the cytoplasmic domains and transmembrane regions. Studies suggest that overexpression of CD98hc may contribute to cell growth and survival by regulating integrin signaling. Studies have shown that CD98hc expression is tightly linked to cell proliferation, and certain antibodies against CD98hc can inhibit cell growth or induce apoptosis in specific cell types.

The present invention exploits the antibody targeting of CD98hc as a means to transport a therapeutic payload across the BBB for the purpose of treating a neurological disease or disorder. Without intending to be limited by any one particular theory or mechanism of action, it is expected that the anti-CD98hc antibody binds to the target receptor on the BBB and is transported to the abluminal side of the BBB. The therapeutic payload contained in the lipid nanoparticle is then expressed in the target tissue.

It is envisaged that any known human CD98hc antibody that effectively targets human CD98hc can be utilised for the purpose of transporting the therapeutic payload across the BBB. In an embodiment, a suitable antibody exhibits one or more or all of the following:
Binding affinity in the range of 10-10,000 nM;
Monovalency or bivalency;
Isoelectric point in the range of 8-9 (Fv isoelectric point between 7.5-9);
No or few post-translational modifications;
Non-competitive binding (does not occlude endogenous ligand binding);
High degree of thermal stability, colloidal stability (not prone to aggregation), freeze-thaw stability and serum stability.

Due to the known similarity of BBB expression levels and high degree of homology between mouse and human CD98hc, it is envisaged that results obtained in mice are translatable to humans. Examples of suitable antibodies include HBJ127 (e.g., Absolute Antibody) and MEM-108 (e.g., ThermoFisher Scientific).

In a preferred embodiment, the CD98hc disclosed herein is human CD98hc (“hCD98hc”) comprising the amino acid sequence as set forth in Uniprot IDs: P08195-1, P08195-2, P08195-3, P08195-4, and P08195-5, the sequences of which are set forth below.

P08195-1 (SEQ ID NO: 1):
MELQPPEASIAVVSIPRQLPGSHSEAGVQGLSAGDDSELGSHCVA
QTGLELLASGDPLPSASQNAEMIETGSDCVTQAGLQLLASSDPPA
LASKNAEVTGTMSQDTEVDMKEVELNELEPEKQPMNAASGAAMSL
AGAEKNGLVKIKVAEDEAEAAAAAKFTGLSKEELLKVAGSPGWVR
TRWALLLLFWLGWLGMLAGAVVIIVRAPRCRELPAQKWWHTGALY
RIGDLQAFQGHGAGNLAGLKGRLDYLSSLKVKGLVLGPIHKNQKD
DVAQTDLLQIDPNFGSKEDFDSLLQSAKKKSIRVILDLTPNYRGE
NSWFSTQVDTVATKVKDALEFWLQAGVDGFQVRDIENLKDASSFL
AEWQNITKGFSEDRLLIAGTNSSDLQQILSLLESNKDLLLTSSYL
SDSGSTGEHTKSLVTQYLNATGNRWCSWSLSQARLLTSFLPAQLL
RLYQLMLFTLPGTPVFSYGDEIGLDAAALPGQPMEAPVMLWDESS
FPDIPGAVSANMTVKGQSEDPGSLLSLFRRLSDQRSKERSLLHGD
FHAFSAGPGLFSYIRHWDQNERFLVVLNFGDVGLSAGLQASDLPA
SASLPAKADLLLSTQPGREEGSPLELERLKLEPHEGLLLRFPYAA
P08195-2 (SEQ ID NO: 2):
MSQDTEVDMKEVELNELEPEKQPMNAASGAAMSLAGAEKNGLVKI
KVAEDEAEAAAAAKFTGLSKEELLKVAGSPGWVRTRWALLLLFWL
GWLGMLAGAVVIIVRAPRCRELPAQKWWHTGALYRIGDLQAFQGH
GAGNLAGLKGRLDYLSSLKVKGLVLGPIHKNQKDDVAQTDLLQID
PNFGSKEDFDSLLQSAKKKSIRVILDLTPNYRGENSWFSTQVDTV
ATKVKDALEFWLQAGVDGFQVRDIENLKDASSFLAEWQNITKGFS
EDRLLIAGTNSSDLQQILSLLESNKDLLLTSSYLSDSGSTGEHTK
SLVTQYLNATGNRWCSWSLSQARLLTSFLPAQLLRLYQLMLFTLP
GTPVFSYGDEIGLDAAALPGQPMEAPVMLWDESSFPDIPGAVSAN
MTVKGQSEDPGSLLSLFRRLSDQRSKERSLLHGDFHAFSAGPGLF
SYIRHWDQNERFLVVLNFGDVGLSAGLQASDLPASASLPAKADLL
LSTQPGREEGSPLELERLKLEPHEGLLLRFPYAA
P08195-3 (SEQ ID NO: 3):
MELQPPEASIAVVSIPRQLPGSHSEAGVQGLSAGDDSGTMSQDTE
VDMKEVELNELEPEKQPMNAASGAAMSLAGAEKNGLVKIKVAEDE
AEAAAAAKFTGLSKEELLKVAGSPGWVRTRWALLLLFWLGWLGML
AGAVVIIVRAPRCRELPAQKWWHTGALYRIGDLQAFQGHGAGNLA
GLKGRLDYLSSLKVKGLVLGPIHKNQKDDVAQTDLLQIDPNFGSK
EDFDSLLQSAKKKSIRVILDLTPNYRGENSWFSTQVDTVATKVKD
ALEFWLQAGVDGFQVRDIENLKDASSFLAEWQNITKGFSEDRLLI
AGTNSSDLQQILSLLESNKDLLLTSSYLSDSGSTGEHTKSLVTQY
LNATGNRWCSWSLSQARLLTSFLPAQLLRLYQLMLFTLPGTPVFS
YGDEIGLDAAALPGQPMEAPVMLWDESSFPDIPGAVSANMTVKGQ
SEDPGSLLSLFRRLSDQRSKERSLLHGDFHAFSAGPGLFSYIRHW
DQNERFLVVLNFGDVGLSAGLQASDLPASASLPAKADLLLSTQPG
REEGSPLELERLKLEPHEGLLLRFPYAA
P08195-4 (SEQ ID NO: 4):
MELQPPEASIAVVSIPRQLPGSHSEAGVQGLSAGDDSELGSHCVA
QTGLELLASGDPLPSASQNAEMIETGSDCVTQAGLQLLASSDPPA
LASKNAEVTETGFHHVSQADIEFLTSIDPTASASGSAGITGTMSQ
DTEVDMKEVELNELEPEKQPMNAASGAAMSLAGAEKNGLVKIKVA
EDEAEAAAAAKFTGLSKEELLKVAGSPGWVRTRWALLLLFWLGWL
GMLAGAVVIIVRAPRCRELPAQKWWHTGALYRIGDLQAFQGHGAG
NLAGLKGRLDYLSSLKVKGLVLGPIHKNQKDDVAQTDLLQIDPNF
GSKEDFDSLLQSAKKKSIRVILDLTPNYRGENSWFSTQVDTVATK
VKDALEFWLQAGVDGFQVRDIENLKDASSFLAEWQNITKGFSEDR
LLIAGTNSSDLQQILSLLESNKDLLLTSSYLSDSGSTGEHTKSLV
TQYLNATGNRWCSWSLSQARLLTSFLPAQLLRLYQLMLFTLPGTP
VFSYGDEIGLDAAALPGQPMEAPVMLWDESSFPDIPGAVSANMTV
KGQSEDPGSLLSLFRRLSDQRSKERSLLHGDFHAFSAGPGLFSYI
RHWDQNERFLVVLNFGDVGLSAGLQASDLPASASLPAKADLLLST
QPGREEGSPLELERLKLEPHEGLLLRFPYAA
P08195-5 (SEQ ID NO: 5):
MELQPPEASIAVVSIPRQLPGSHSEAGVQGLSAGDDSETGSDCVT
QAGLQLLASSDPPALASKNAEVTVETGFHHVSQADIEFLTSIDPT
ASASGSAGITGTMSQDTEVDMKEVELNELEPEKQPMNAASGAAMS
LAGAEKNGLVKIKVAEDEAEAAAAAKFTGLSKEELLKVAGSPGWV
RTRWALLLLFWLGWLGMLAGAVVIIVRAPRCRELPAQKWWHTGAL
YRIGDLQAFQGHGAGNLAGLKGRLDYLSSLKVKGLVLGPIHKNQK
DDVAQTDLLQIDPNFGSKEDFDSLLQSAKKKSIRVILDLTPNYRG
ENSWFSTQVDTVATKVKDALEFWLQAGVDGFQVRDIENLKDASSF
LAEWQNITKGFSEDRLLIAGTNSSDLQQILSLLESNKDLLLTSSY
LSDSGSTGEHTKSLVTQYLNATGNRWCSWSLSQARLLTSFLPAQL
LRLYQLMLFTLPGTPVFSYGDEIGLDAAALPGQPMEAPVMLWDES
SFPDIPGAVSANMTVKGQSEDPGSLLSLFRRLSDQRSKERSLLHG
DFHAFSAGPGLFSYIRHWDQNERFLVVLNFGDVGLSAGLQASDLP
ASASLPAKADLLLSTQPGREEGSPLELERLKLEPHEGLLLRFPYA
A

Alternatively, an anti-CD98hc antibody or antigen-binding fragment thereof (such as a VHH or scFv fragment) can be produced using suitable methods in the art in view of the present disclosure. For example, a VHH or scFv fragment can be recombinantly produced by growing a recombinant host cell (such as a bacterial, yeast or mammalian cell) under suitable conditions for the production of the antibody fragment and recovering the fragment from the cell culture. Assessment of the binding characteristics of the antibody can also determined using known methods in the art. For example, antibody affinity may be measured by a radiolabeled antigen binding assay, performed with a Fab version of an antibody of interest and its antigen. For example, solution binding affinity of Fabs for antigen is measured by equilibrating Fab with a minimal concentration of (125 I)-labeled antigen in the presence of a titration series of unlabeled antigen, then capturing bound antigen with an anti-Fab antibody-coated plate (see, e.g., Chen et al., J Mal. Biol. 293:865-881(1999)).

For example, a surrogate measurement for the affinity of one or more antibodies for the BBB-R is its half maximal inhibitory concentration (IC50), a measure of how much of the antibody is needed to inhibit the binding of a known BBB-R ligand to the BBB-R by 50%. Several methods of determining the IC50 for a given compound are art-known; a common approach is to perform a competition binding assay. In general, a high IC50 indicates that more of the antibody is required to inhibit binding of the known ligand, and thus that the antibody's affinity for that ligand is relatively low. Conversely, a low IC50 indicates that less of the antibody is required to inhibit binding of the known ligand, and thus that the antibody's affinity for that ligand is relatively high.

An exemplary competitive ELISA assay to measure IC50 is one in which increasing concentrations of anti-CD98h antibodies are used to compete against biotinylated anti-CD98hc antibody for binding to CD98hc. The anti-CD98hc competition ELISA is performed in Maxisorp plates (Neptune, NJ.) coated with 2.5 μg/ml of purified murine CD98hc extracellular domain in PBS at 4° C. overnight. Plates are washed with PBS/0.05%>Tween 20 and blocked using Superblock blocking buffer in PBS (Thermo Scientific, Hudson, NH). A titration of each individual anti-CD98hc (1:3 serial dilution) can be combined with biotinylated anti-CD98hc (0.5 nM final concentration) and added to the plate for 1 hour at room temperature. Plates are washed with PBS/0.05% Tween 20, and HRP-streptavidin (Southern Biotech, Birmingham) is added to the plate and incubated for 1 hour at room temperature. Plates were washed with PBS/0.05% Tween 20, and biotinylated anti-CD98hc bound to the plate is detected using TMB substrate (BioFX Laboratories, Owings Mills). Antibody binding affinity can also be measured via surface plasmon resonance (SPR) and FACS (FACS for cell based binding experiments).

In certain embodiments, the CD98hc is glycosylated. In certain embodiments, the CD98hc is phosphorylated.

The terms anti-CD98hc antibody and an antibody that binds to CD98hc refer to an antibody that is capable of binding CD98hc.

In an embodiment, antibody binding to CD98hc does not inhibit amino acid transport by the CD98 heterodimeric complex, and optionally does not inhibit cell growth, cell adhesion, proliferation and/or apoptosis. In other words, the CD98hc antibody can utilize transcytosis across the BBB without otherwise affecting the physiological role of LAT1 or LAT2. In vitro assays which may be used to detect amino acid transport by CD98hc (e.g., in a heterodimeric complex with a CD98 light chain (e.g., LAT1, LAT2, y+LAT1, y+LAT2, xCT, and asc-1) are known in the art. See, e.g., Fenczik, C. A et al. (2001) J. Biol. Chem. 276, 8746-8752; see also, US 2013/0052197. Further, assays for measuring the effect of a CD98hc-binding antibody on cell proliferation, cell division, apoptosis and cell adhesion can be performed, by way of example and without limitation, according to the methods described in U.S. 2013/0052197. See also, Yagita H. et al. (1986) Cancer Res. 46: 1478-1484; and Warren A P., et al. (1996) Blood 87:3676-3687.

In an embodiment, the kinetics of amino acid transport of any of the native ligands of the CD98 heterodimeric complex across the BBB in the presence of the anti-CD98hc antibody can be determined compared to the kinetics in the absence of the antibody, wherein the kinetics in the absence of the antibody are one or more of:
KM=295 μM for glutamine (in the presence of NaCl);
KM=236 μM for leucine (in the presence of NaCl);
KM=120 μM for arginine (in the presence of NaCl); and
KM=138 μM for arginine (in the absence of NaCl).

The amino acid transport kinetics of the CD98 amino acid transporter can be measured in an assay as described, e.g., by Broer et al., Biochem J. 2000 Aug. 1; 349 Pt 3:787-95.

In certain embodiments, an antibody provided herein is an antibody fragment. Antibody fragments include, but are not limited to, Fab, Fab′, Fab′-SH, F(ab′)2, Fv, and scFv fragments, and other fragments described below. For a review of certain antibody fragments, see Hudson et al. Nat. Med 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat. Med 9: 129-134 (2003); and Hollinger et al., Proc. Natl. Acad Sci. USA 90: 6444-6448 (1993). Triabodies and tetrabodies are also described in Hudson et al., Nat. Med 9: 129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (see, e.g., U.S. Pat. No. 6,248,516 B1). Antibody fragments can be made by various techniques, including but not limited to proteolytic digestion of an intact antibody as well as production by recombinant host cells (e.g. E. coli or phage).

In certain embodiments, an antibody provided herein is a chimeric antibody. Certain chimeric antibodies are described, e.g., in U.S. Pat. No. 4,816,567; and Morrison et al., Proc. Natl. Acad Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13: 1619-1633 (2008), and are further described, e.g., in Riechmann et al., Nature 332:323-329 (1988); Queen et al., Proc. Nat Acad Sci. USA 86: 10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mal. Immunol. 28:489-498 (1991) (describing “resurfacing”); Dall'Acqua et al., Methods 36:43-60 (2005) (describing “FR shuffling”); and Osboum et al., Methods 36:61-68 (2005) and Klimka et al., Br. J Cancer, 83:252-260 (2000) (describing the “guided selection” approach to FR shuffling).

Human framework regions that may be used for humanization include but are not limited to: framework regions selected using the “best-fit” method (see, e.g., Sims et al. J Immunol. 151:2296 (1993)); framework regions derived from the consensus sequence of human antibodies of a particular subgroup of light or heavy chain variable regions (see, e.g., Carter et al. Proc. Natl. Acad Sci. USA, 89:4285 (1992); and Presta et al. J Immunol., 151:2623 (1993)); human mature (somatically mutated) framework regions or human germline framework regions (see, e.g., Almagro and Fransson, Front. Biosci. 13: 1619-1633 (2008)); and framework regions derived from screening FR libraries (see, e.g., Baca et al., J Biol. Chem. 272: 10678-10684 (1997) and Rosok et al., J Biol. Chem. 271:22611-22618 (1996)).

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008). Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extra-chromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23: 1117-1125 (2005). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J Immunol., 133: 3001 (1984); Brodeur et al., Monoclonal Antibody Production Techniques and Applications, pp. 51-63. Human antibodies generated via human B-cell hybridoma technology are also known (Li et al., Proc Natl Acad Sci USA; 103: 3557-3562). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3): 185-91 (2005). Human antibodies may also be generated by isolating Fv clone variable domain sequences selected from human-derived phage display libraries. Such variable domain sequences may then be combined with a desired human constant domain.

Antibodies for use in the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178: 1-37 (O'Brien et al., ed., Human Press, Totowa, NJ, 2001) and further described, e.g., in the McCafferty et al., Nature 348:552-554; Clackson et al., Nature 352: 624-628 (1991).

In certain phage display methods, repertoires of VH and VL genes are separately cloned by PCR and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the repertoire can be cloned (e.g., from human) to provide a single source of antibodies to a wide range of non-self and also self antigens without any immunization as described by Griffiths et al., EMBO J, 12: 725-734 (1993). Finally, naïve libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J Mal. Biol., 227: 381-388 (1992). Antibodies or antibody fragments isolated from human antibody libraries are considered human antibodies or human antibody fragments herein.

### Lipid Nanoparticles

In an embodiment of the invention, a nucleic acid of the invention may be formulated in a liposome, lipoplex or a lipid nanoparticle so as to increase stability and transfection of the mRNA. Liposomes are artificially-prepared vesicles which are primarily composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter.

Liposomes, lipoplexes or lipid nanoparticles may include, but are not limited to, opsonins or ligands in order to improve the attachment of liposomes, lipoplex or lipid nanoparticles to different tissues or to activate events such as, but not limited to, endocytosis and transcytosis. Liposomes, lipoplexes or lipid nanoparticles may contain a low or a high pH in order to improve the delivery of the pharmaceutical formulations. The formation of liposomes, lipoplexes or lipid nanoparticles may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the size, polydispersity and the shelf-life of the vesicles for the intended application, and the batch-to-batch reproducibility and possibility of large-scale production of safe and efficient liposome, lipoplex or lipid nanoparticle products.

Preferably, the mRNA of the invention is formulated in a lipid nanoparticle. LNPs are composed primarily of ionizable along with other lipid ingredients. These typically include “helper lipids” such as neutral phospholipid molecules belonging to the phosphatidylcholine (PC) or phosphoethanolamine (PE) class, or charged lipids such as DOTAP or DOTMA, and sterols, such as cholesterol. As used herein, the term “helper lipid” is a term understood in the art to be a lipid that increases one or more or all of particle stability, fluidity, tissue targeting specificity, and/or transfection efficiency. Another common lipid ingredient is known as a PEGylated phospholipid—a polyethylene glycol (PEG) polymer covalently attached to the head-group of a phospholipid. Alternatively, a surfactant such as Polysorbate 20 or Polysorbate 80 can be used. In yet another embodiment, the surfactant may be selected from the group consisting of a non-ionic surfactant, an anionic surfactant, a cationic surfactant or an amphoteric surfactant.

As used herein, the term lipid nanoparticle (LNP) shall therefore be understood to refer to lipid-based particles having at least one dimension in the order of nanometers (e.g., 1-1,000 nm) and which typically comprise a nucleic acid as described herein. In embodiments, LNPs are formulated in a composition for delivery of a nucleic acid to a desired target across the BBB. For example, the LNP may be any lipid composition, including, but not limited to, liposomes or vesicles, where an aqueous volume is encapsulated by amphipathic lipid bilayers (e.g., single; unilamellar or multiple; multilamellar), micelle-like lipid nanoparticles having a non-aqueous core and solid lipid nanoparticles, wherein solid lipid nanoparticles lack lipid bilayers.

Whilst liposomes include one or more rings of lipid bilayer surrounding an aqueous pocket, not all LNPs have a contiguous bilayer like liposomes. Instead, it is understood that some LNPs assume a micelle-like structure, encapsulating drug molecules in a non-aqueous core. Suitable cationic lipids may include those described in the cationic lipid may be selected from, but not limited to, a cationic lipid described in International Publication Nos. WO2012040184, WO2011153120, WO2011149733, WO2011090965, WO2011043913, WO2011022460, WO2012061259, WO2012054365, WO2012044638, WO2010080724, WO201021865, WO2008103276, WO2013086373 and WO2013086354, U.S. Pat. Nos. 7,893,302, 7,404,969, 8,283,333, and 8,466,122 and US Patent Publication No. US20100036115, US20120202871, US20130064894, US20130129785, US20130150625, US20130178541 and US20130225836. Other suitable cationic lipids, non-cationic lipids, PEG lipids and structural lipids, and suitable ratios thereof include those disclosed in WO 2015164674 and WO 2013090648.

Exemplary LNP compositions and methods of making same are described, for example, in Semple et al., Nature Biotechnology, 28: 172-176 (2010); and Jayarama et al., Angew Chem Int Ed Engl 51(34): 8529-33 (2012). Alternatively, the LNP formulation may be formulated by the methods described in WO2011127255 or WO2008103276.

Further, the particle size of the lipid nanoparticle may be increased and/or decreased. The change in particle size may be able to help counter biological reaction such as, but not limited to, inflammation or may increase the biological effect of the nucleic acid delivered to a given subject.

LNPs suitable for use in the present disclosure will be apparent to the skilled person and/or are described herein. The lipids can have an anionic, cationic or zwitterionic hydrophilic head group. In one embodiment, the lipid nanoparticle comprises a PEG-lipid, a sterol structural lipid and/or a neutral lipid.

In an embodiment, the LNP encapsulates the nucleic acid. In another embodiment, the nucleic acid is bound to the LNP. In another embodiment, the nucleic acid is adsorbed on to the LNP.

In an embodiment, the LNP comprises an ionizable lipid, a helper lipid, sterol, and PEG-lipid. In another embodiment, the LNP comprises an ionizable lipid, a helper lipid, sterol, and a surfactant. In an embodiment, the molar ratio of ionizable lipid, a helper lipid, sterol, and PEG-lipid, or surfactant, is 50:10:38.5:1.5. In an embodiment, the molar ratio of ionizable lipid, a helper lipid, sterol, and PEG-lipid or surfactant is selected from the group consisting of 60:5:10:25, 55:30:45:0.2, 52:8:38.5:1.5, 52:8:37:3, 50:20:23.5:6.5 50:10.5:38:1.5, 50:12.5:35:2.5, 45:13:39.5:2.5, 35:16:46.5:2.5, 35:40:22.5:2.5, 26.5:20:52:1.5, 25:30:30:1, 40:10:38.5:1.5, 30:10:38.5:1.5, 40:10:38.5:1.5, 60:10:38.5:1.5, 70:10:38.5:1.5, 50:5:38.5:1.5, 50:15:38.5:1.5, 50:20:38.5:1.5, 50:25:38.5:1.5, 50:10:18.5:1.5, 50:10:28.5:1.5, 50:10:48.5:1.5, 50:10:58.5:1.5, 50:10:38.5:0.5, 50:10:38.5:1.0, 50:10:38.5:2.0, 50:10:38.5:2.5 or any combination thereof.

In an embodiment, the lipid nanoparticle comprises 20-60 mol % (e.g., 20-30 mol %, 20-40 mol %, 20-50 mol %, 20-60 mol %, 30-40 mol %, 30-50 mol %, 30-60 mol %, 40-50 mol %, 40-60 mol %, 45-55 mol %, or 45-50 mol % or 50-60 mol %) ionizable lipid; 5-25 mol % (e.g., 5-10 mol %, 5-15 mol %, 5-20 mol %, 5-25 mol %, or 10-15 mol %, 10-20 mol %, 10-25 mol %, 15-20 mol %, 15-25 mol %) a helper lipid; 25-55 mol % (e.g., 25-35 mol %, 25-45 mol %, 25-55 mol %, 35-45 mol %, 35-55 mol % or 45-55 mol %) sterol; and 0.5-15 mol % (e.g., 0.5-5 mol %, 0.5-10 mol %, or 0.5-15 mol %, 2.5-5 mol %, 2.5-10 mol %, 2.5-15 mol %, 5-10 mol %, 5-15 mol %) PEG-lipid or surfactant.

In an embodiment, the ionizable lipid is selected from the group consisting of 9-Heptadecanyl 8-{(2-hydroxyethyl)[6-oxo-6-(undecyloxy)hexyl]amino}octanoate (SM-102), DLin-MC3-DMA (MC3), 1,2-distearyloxy-N,N-dimethyl-3-aminopropane (DSDMA), 1,2-dioleyloxy-N,Ndimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA), 2,5-bis((9z,12z)-octadeca-9,12,dien-1-yloxyl)benzyl-4-(dimethylamino)butnoate (LKY750), 4-hydroxybutyl)azanediyl)bis(hexane-6,1-diyl)bis(2-hexyldecanoate (ALC0315), C12-200, 306-O12B, 4A3-SC8, cKK-E12, 2,2-dilinoleyl-4-dimethylaminoethyl-[1,3]-dioxolane (DLin-KC2-DMA), and di((Z)-non-2-en-1-yl) 9-((4-(dimethylamino)butanoyl)oxy)heptadecanedioate (L319). Preferably the ionizable lipid is SM-102 or MC3.

In an embodiment, the helper lipid is a cationic lipid. In this embodiment, the cationic lipid is selected from the group consisting of 1,2-dioleoyl-3-trimethylammonium-propane (DOTAP), 1,2-dioleoyl-s-glycero-3-ethylphosphocholine (EPC), dimethyldioctadecylammonium bromide (DDAB), and 1,2-di-O-octadecenyl-3-trimethylammonium-propane (DOTMA). In another embodiment, the helper lipid is a phospholipid. In this embodiment, the phospholipid is selected from the group consisting of 1,2-distearoyl-sn-glycero-3-phosphocholine (DSPC), 1,2-dioleoyl-3-trimethylammonium-propane (DOTAP), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OchemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine (DSPE), 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin and combinations thereof. Preferably, the phospholipid is DSPC or DOPE.

In an embodiment, the sterol is selected from the group consisting of β-sitosterol, cholesterol, 24(S)-hydroxycholesterol, 20α-hydroxycholesterol, cholesterol oleate, other cholesterol esters, fecosterol, campesterol, stigmasterol, brassicasterol, ergosterol, tomatidine, tomatine, ursolic acid and alpha-tocopherol and combinations thereof. Preferably, the sterol is β-sitosterol or cholesterol.

In another embodiment, the PEG-lipid is selected from the group consisting of PEG2000-c-DMG, PEG2000-DMG, PEG2000-DLPE, PEG2000-DMPE, PEG2000-DPPC, a PEG2000-DSPE lipid and combinations thereof. Preferably, the PEG2000-lipid is PEG2000-DSPE or PEG2000-DMG. In another embodiment, the PEG-lipid may be a different molecular weight as whose suitability is determined by a skilled person in the art. For example, the PEG-lipid may have a molecular weight of 200, 400, 600, 800, 1000, 1200, 1400, 1600, 1800, 2000, 2200, 2400, 2600, 2800, 3000, 3200, 3400, 3600 or 4000.

In an embodiment, the surfactant is selected from the group consisting of a nonionic surfactant, an anionic surfactant, a cationic surfactant or an amphoteric surfactant. In an embodiment, the surfactant is Polysorbate 20 or Polysorbate 80.

In an embodiment, the LNP further comprises one or more additional helper lipids. For example, the LNP may comprise an additional permanently cationic or anionic lipid described herein or known in the art, or another lipid component such as but not limited to ceramide, sphingosine, sphingomyelin, cerebroside, LPC oleate, alpha-tocopherol, folate-conjugated lipid, dehydroascorbic acid-conjugated lipid or 6-O-glucose oleate-conjugated lipid.

In one example, the pharmaceutical composition of the present disclosure further comprises a polymeric microparticle.

The skilled person will be aware that various polymers can form microparticles to encapsulate or adsorb the nucleic acid of the present disclosure. It will be apparent that use of a substantially non-toxic polymer means that particles are safe, and the use of a biodegradable polymer means that the particles can be utilised after delivery to avoid long-term persistence. Useful polymers are also utilised to assist in the preparation of pharmaceutical grade formulations.

Exemplary non-toxic and biodegradable polymers include, but are not limited to, poly(a-hydroxy acids), polyhydroxy butyric acids, polylactones (including polycaprolactones), polydioxanones, polyvalerolactone, polyorthoesters, polyanhydrides, polycyanoacrylates, tyrosine-derived polycarbonates, polyvinyl-pyrrolidinones or polyester-amides, and combinations thereof.

In an embodiment, the pharmaceutical composition of the present disclosure further comprises an oil-in-water cationic emulsion. Suitable oils for use in an oil-in-water emulsion will be apparent to the skilled person and/or are described herein. For example, the emulsion comprises one or more oils derived, for example, from an animal (e.g., fish) or a vegetable source (e.g., nuts, seeds, grains). The skilled person will understand that biocompatible and biodegradable oils are preferentially used. Exemplary animal oils (i.e., fish oils) include cod liver oil, shark liver oils, and whale oil. Exemplary vegetable oils include peanut oil, coconut oil, olive oil, soybean oil, jojoba oil, safflower oil, cottonseed oil, sunflower seed oil, sesame seed oil, corn oil.

In addition to the oil, the oil-in-water emulsion also comprises a cationic lipid to facilitate formation and stabilisation of the emulsion. Suitable cationic lipids will be apparent to the skilled person and/or are described herein. Exemplary cationic lipids include, but are not limited to, limited to: 1,2-dioleoyloxy-3-(trimethylammonio)propane (DOTAP), 3′-[N—(N′,N′-Dimethylaminoethane)-carbamoyl] Cholesterol (DC Cholesterol), dimethyldioctadecyl-ammonium (DDA), 1,2-Dimyristoyl-3-Trimethyl-AmmoniumPropane (DMTAP), dipalmitoyl[C16:0]trimethyl ammonium propane (DPTAP) and distearoyltrimethylammonium propane (DSTAP).

In some embodiments, the oil-in-water emulsion also comprises a non-ionic surfactant and/or a zwitterionic surfactant. The skilled person will be aware of surfactants suitable for use in the present disclosure. Exemplary surfactants include, but are not limited to: the polyoxyethylene sorbitan esters surfactants (e.g., polysorbate 20 and polysorbate 80) and copolymers of ethylene oxide (EO), propylene oxide (PO), and/or butylene oxide (BO).

As delivery across the blood brain barrier is contemplated, the formulation may further comprise one or more components to aid delivery across the blood brain barrier. In an embodiment, there is therefore provided LNP formulations that additionally comprise a permeability enhancer molecule. Non-limiting permeability enhancer molecules are described in US Patent Publication No. US20050222064.

### Nucleic Acid Cargos

In an aspect of the invention, there is provided a nucleic acid that encodes an intracellular lipid trafficker that includes a codon optimisation and/or chemical modification. In a preferred embodiment the nucleic acid is messenger ribonucleic acid (mRNA) encoding a therapeutic polypeptide but may otherwise be selected from the group consisting of deoxyribonucleic acid (DNA), small interfering RNA (siRNA), antisense oligonucleotide (ASO), small hairpin RNA (shRNA), micro-RNA (miRNA) or long non-coding RNA (lncRNA).

mRNA is a single-stranded molecule of ribonucleic acid (RNA) that corresponds to the genetic sequence of a gene. mRNA is created during the process of transcription, where an enzyme (RNA polymerase) converts the gene into primary transcript mRNA (also known as pre-mRNA).

In accordance with the invention, the term messenger RNA (mRNA) refers to a polynucleotide which encodes a polypeptide of interest and which is capable of being translated to produce an encoded polypeptide in vitro, in vivo, in situ or ex vivo, preferably in vivo. The term “mRNA” may be used interchangeably with “mRNA molecule” herein.

In producing a mRNA of the invention, a skilled person would understand that mRNA sequences can be synthesised according to the methods described herein or known in the art including those described in the Example. It is envisaged that the mRNA sequence is optimised by in vitro transcription from a DNA template and that the DNA sequence encoding the mRNA is codon optimised, preferably enriched for G and C bases.

In an embodiment, the mRNA sequence is then chemically modified to increase mRNA stability and/or mRNA translation when compared to a mRNA without chemical modification. For example, the chemical modification may be a nucleoside modification whereby a base is modified to a uracil (U) or a cytosine but the modification may be to any mRNA base including adenine (A), and guanine (G) if the modification increases mRNA stability and/or mRNA translation when compared to a mRNA without chemical modification.

In an embodiment, the “corresponding mRNA” may be a mRNA without the chemical and/or nucleoside modification which may also be referred to as a wild-type mRNA. A “wildtype mRNA” refers to any mRNA wild-type gene that is capable of having normal (level of function absent disease or disorder) biological activity when expressed as a protein in vivo.

Where it is contemplated that a mRNA of the invention provides for restored cellular function, this is typically understood to mean that the mRNA has restored the cells' capacity in a manner that approximates the effect of a wild-type polypeptide, however in some cases, it may be about 70%, about 75%, about 80%, about 85%, about 90%, about 95%, about 96%, about 97%, about 98%, about 99% or about 99.5% or higher when compared to a wild-type polypeptide.

In an embodiment, the coding region of the mRNA molecule or the region that encodes a polypeptide may comprise from about 100 to about 200, from about 200 to about 300, from about 300 to about 400, from about 400 to about 500, from about 500 to about 600, from about 600 to about 700, from about 700 to about 800, from about 800 to about 900, from about 900 to about 1000, from about 1000 to about 1200, from about 1200 to about 1400, from about 1400 to about 1600, from about 1600 to about 1800, from about 1800 to about 2000, from about 2000 to about 2200, from about 2200 to about 2400, from about 2400 to about 2600, from about 2600 to about 2800, from about 2800 to about 3000, from about 3000 to about 3200, from about 3200 to about 3400, from about 3400 to about 3600, from about 3600 to about 3800, from about 3800 to about 4000, from about 4000 to about 4200, from about 4200 to about 4400 or more nucleotides or bases.

It is understood that the basic components of an mRNA molecule include at least a 5′ cap, a 5′UTR, a coding region, a 3′UTR and a poly-A tail (in 5′ to 3′ direction) and has optionally been codon optimised to provide for enrichment of guanine (G) and cytosine bases. The mRNA may also comprise an optimised codon and/or a chemical modification A skilled person will understand that similar optimisation can be made to other mRNA sequences or molecules for the purpose of increasing or restoring protein expression, enhancing mRNA stability, thermal stability or functionality. Such methods may include those described herein including in the Example, or known in the art.

### Untranslated Regions

5′UTRs are regions of a gene that are transcribed but not translated. In mRNA, the 5′UTR starts at the transcription start site and continues to the start codon but does not include the start 5 codon; whereas the 3′UTR starts immediately following the stop codon and continues until the end of the transcript. 5′UTRs play a role in stability and translation initiation. They harbor signatures like Kozak sequences which are commonly known to be involved in the process by which the ribosome initiates translation of many genes. Kozak sequences have the consensus CCR(NG)CCAUGG, where R is a purine (adenine or guanine) three bases upstream of the start codon (AUG), which is followed by another ‘G’. 5′UTR also have been known to form secondary structures which are involved in elongation factor binding.

By engineering the features typically found in abundantly expressed genes of specific target organs, one can enhance the stability and protein production of mRNA of the invention. For example, introduction of 5′ UTR of liver-expressed mRNA, such as albumin, serum amyloid A, Apolipoprotein NB/E, transferrin, alpha fetoprotein, erythropoietin, or Factor VIII, could be used to enhance expression of a nucleic acid molecule, such as a mRNA, in hepatocytes or other liver cells. Likewise, use of 5′ UTR from other tissue-specific mRNA to improve expression in that tissue is possible for muscle (MyoD, Myosin, Myoglobin, Myogenin, Herculin), for endothelial cells (Tie-I, CD36), for myeloid cells (C/EBP, AMLI, G-CSF, GM-CSF, CDllb, MSR, Fr-1, i-NOS), for leukocytes (CD45, CD18), for adipose tissue (CD36, GLUT4, ACRP30, adiponectin) and for lung epithelial cells (SP-NB/CID). Other 5′UTRs are well known in the art.

Other non-UTR sequences may be incorporated into the 5′ (or 3′ UTR) UTRs. For example, introns or portions of intron sequences, or RNA aptamers may be incorporated into the flanking regions of the polynucleotides, primary constructs or mRNA of the invention. Incorporation of intronic sequences may increase protein production as well as mRNA levels.

3′ UTRs are known to have stretches of uridines embedded in them. These AU rich signatures are particularly prevalent in genes with high rates of turnover. Based on their sequence features and functional properties, the AU rich elements (AREs) can be separated into three classes: Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-μ. Class III ARES are less well defined. These U rich regions do not contain an AUUUA motif C-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Therefore, it is envisaged that HuR specific binding sites may be engineered into the 3′ UTR of nucleic acid molecules which will lead to HuR binding and thus, stabilization of the message in vivo.

Introduction, removal or modification of 3′ UTR AU rich elements (AREs) can be used to modulate the stability of mRNA of the invention. Although less preferable, when engineering specific mRNA, one or more copies of an ARE can be introduced to make mRNA of the invention less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using mRNA of the invention and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection. Suitable 3′UTRs are well known in the art.

### 5′ Capping

The 5′ cap structure of an mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal intrans removal during mRNA splicing.

Endogenous mRNA molecules may be 5′-end capped generating a 5′-ppp-5′-triphosphate linkage between a terminal guanosine cap residue and the 5′-terminal transcribed sense nucleotide of the mRNA molecule. This 5′-guanylate cap may then be methylated to generate an NY-methyl-guanylate residue. The ribose sugars of the terminal and/or anteterminal transcribed nucleotides of the 5′ end of the mRNA may optionally also be 2′-O-methylated. 5′-decapping through hydrolysis and cleavage of the guanylate cap structure may target a nucleic acid molecule, such as an mRNA molecule, for degradation.

Modifications to the mRNA of the present invention may generate a non-hydrolysable cap structure preventing decapping and thus increasing mRNA half-life. Because cap structure hydrolysis requires cleavage of 5′-ppp-5′ phosphorodiester linkages, modified nucleotides may be used during the capping reaction. For example, a Vaccinia Capping Enzyme from New England Biolabs (Ipswich, MA) may be used with μ-thio-guanosine nucleotides according to the manufacturer's instructions to create a phosphorothioate linkage in the 5′-ppp-5′ cap. Additional modified guanosine nucleotides may be used such as a-methyl-phosphonate and seleno-phosphate nucleotides.

Additional modifications include, but are not limited to, 2′-O-methylation of the ribose sugars of 5′-terminal and/or 5′-anteterminal nucleotides of the mRNA on the 2′-hydroxyl group of the sugar ring. Multiple distinct 5′-cap structures can be used to generate the 5′-cap of a nucleic acid molecule, such as an mRNA molecule.

Cap analogs, or synthetic cap analogs, chemical caps, chemical cap analogs, or structural or functional cap analogs, differ from natural (i.e. endogenous, wild-type or physiological) 5′-caps in their chemical structure, while retaining cap function. Cap analogs may be chemically (i.e. non-enzymatically) or enzymatically synthesized and/or linked to a nucleic acid molecule. For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m7G-3′mppp-G; which may equivalently be designated 3′ O-Me-m7G(5′)ppp(5′)G). The 3′-O atom of the other, unmodified, guanine becomes linked to the 5′-terminal nucleotide of the capped nucleic acid molecule (e.g. an mRNA or mmRNA). The N7- and 3′-O-methlyated guanine provides the terminal moiety of the capped nucleic acid molecule (e.g. mRNA or mmRNA). Another cap is mCAP, which is similar to ARCA but has a 2′-O-methyl group on guanosine (i.e., N7,2′-O-dimethyl-guanosine-5′triphosphate-5′-guanosine, m7Gm-ppp-G).

While cap analogs allow for the concomitant capping of a nucleic acid molecule in an in vitro transcription reaction, up to 20% of transcripts can remain uncapped. This, as well as the structural differences of a cap analog from an endogenous 5′-cap structures of nucleic acids produced by the endogenous, cellular transcription machinery, may lead to reduced translational competency and reduced cellular stability.

mRNA of the invention may also be capped post-transcriptionally, using enzymes, in order to generate a cap representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′-cap structures are those which, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′triphosphate linkage between the 5′-terminal nucleotide of an mRNA and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to 7mG(5′)ppp(5′)N,pN2p (cap 0), 7mG(5′)ppp(5′)NlmpNp (cap 1), and 7mG(5′)ppp(5′)NlmpN2mp (cap 2).

Because the mRNA may be capped post-transcriptionally, and because this process is more efficient, it is preferred that the mRNA be capped. 5′ terminal caps may include endogenous caps or cap analogs. A 5′ terminal cap may comprise a guanine analog. Useful guanine analogs include, but are not limited to, inosine, N1-methyl-guanosine, 2′fluoro-0 guanosine, 7-deaza-guanosine, 8-oxo-guanosine, 2-amino-guanosine, LNA-guanosine, and 2-azido-guanosine.

A suitable non-limiting example of a cap structure that is useful in the invention is a Cap1 structure, preferably the sequence as set forth in the sequence, AG. A skilled person will be able to determine other suitable cap structures useful in the invention.

### IRES Sequences

Further provided are mRNA which may contain an internal ribosome entry site (IRES). IRES plays an important role in initiating protein synthesis in absence of the 5′ cap structure. An IRES may act as the sole ribosome binding site, or may serve as one of multiple ribosome binding sites of an mRNA. mRNA containing more than one functional ribosome binding site may encode several peptides or polypeptides that are translated independently by the ribosomes (“multicistronic nucleic acid molecules”). When mRNA are provided with an IRES, further optionally provided is a second translatable region. Examples of IRES sequences that can be used according to the invention include without limitation, those from picornaviruses (e.g. FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

### Poly-A Tails

During RNA processing, a long chain of adenine nucleotides (poly-A tail) may be added to a polynucleotide such as an mRNA molecule in order to increase stability. Immediately after transcription, the 3′ end of the transcript may be cleaved to free a 3′ hydroxyl. Then poly-A polymerase adds a chain of adenine nucleotides to the RNA. The process, called polyadenylation, adds a poly-A tail that can be between, for example, approximately 100 and 250 residues long.

Generally, the length of a poly-A tail of the present invention is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the polynucleotide or mRNA includes a poly-A tail from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In another embodiment, the poly A tail comprises between 25 to 200 nucleotides, between 50 to 150 nucleotides, between 80 to 120 nucleotides, preferably about 100 nucleotides.

In one embodiment, the poly-A tail is designed relative to the length of the overall mRNA. This design may be based on the length of the coding region, the length of a particular feature or region (such as the first or flanking regions) or based on the length of the ultimate product expressed from the mRNA. In this context the poly-A tail may be 10, 20, 30, 40, 50, 60, 70, 80, 90, or 100% greater in length than the polynucleotides or mRNA or feature thereof. The poly-A tail may also be designed as a fraction of mRNA to which it belongs. In this context, the poly-A tail may be 10, 20, 30, 40, 50, 60, 70, 80, or 90% or more of the total length of the construct or the total length of the construct minus the poly-A tail. Further, engineered binding sites and conjugation of mRNA for Poly-A binding protein may enhance expression.

Additionally, multiple distinct mRNA may be linked together to the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In one embodiment, a mRNA of the present invention may be designed to include a polyA-G Quartet. The G-quartet is a cyclic hydrogen bonded array of four guanine nucleotides that can be formed by G-rich sequences in both DNA and RNA. In this embodiment, the G-quartet is incorporated at the end of the poly-A tail. The resultant mRNA construct is assayed for stability, protein production and other parameters including half-life at various time points. It has been discovered that the polyA-G quartet results in protein production equivalent to at least 75% of that seen using a poly-A tail of 120 nucleotides alone.

### Codon Optimisation

A nucleotide sequence of a mRNA useful for the invention may be codon optimised. In performing codon optimisation, codon frequencies in target and host organisms are generally matched to ensure proper folding, bias GC content to increase mRNA stability or reduce secondary structures, minimize tandem repeat codons or base runs that may impair gene construction or expression, customize transcriptional and translational control regions, insert or remove protein trafficking sequences, remove/add post translation modification sites in encoded protein (e.g., glycosylation sites), add, remove or shuffle protein domains, insert or delete restriction sites, modify ribosome binding sites and mRNA degradation sites, to adjust translational rates to allow the various domains of the protein to fold properly, or to reduce or eliminate problem secondary structures within the mRNA.

Codon composition is known to affect translation efficiency. Replacing rare codons with synonymous frequent codons improves translational yield because reuse of the same tRNA accelerates translation owing to amino-acylation of tRNAs in the vicinity of the ribosomes. Codon context (that is, neighboring nucleotides and codons) also affects the translational elongation rate and translational efficiency. Similar to recombinant DNA-based approaches, codon-optimised in vitro transcribed (IVT) mRNAs have been successfully used. However, in some cases, there may be valid reasons to refrain from using optimised codons as understood by a skilled person. Some proteins require slow translation, which is ensured by rare codons, for their proper folding. It may also be beneficial for some IVT mRNA-encoded to maintain the original ORF.

Codon optimisation methods are useful to increase expression, structural stability, thermal stability or increased function of the encoded protein. Codon optimisation tools, algorithms and services are known in the art, and non-limiting examples include services from GeneArt (Life Technologies), DNA2.0 (Menlo Park CA) or other methods known in the art. However, specific strategies for codon optimisation vary considerably based on underlying assumptions about which codon features are important to translation. One approach involves substituting in the most frequently used codon for all instances of a given amino acid. Another approach involves only replacing rare codons with more abundant synonymous codons. Still other approaches involve adjusting the codon usage frequency to match the natural frequencies in a host organism, or choosing codons based on cognate transfer RNA (tRNA) abundance. A skilled person will generally understand that codon optimisation involves the replacement of a codon with an optimised codon that is synonymous with the replaced codon.

In one embodiment, the ORF sequence is optimised using optimisation algorithms. In a preferred embodiment, codon optimisation is conducted by enriching a DNA template that will encode a mRNA of the invention for guanine (G) and cytosine content. Even more preferably, G and C content is enriched by substituting codons with adenine (A) or uracil (U) at the third base with codons enriched in guanine (G) or cytosine (C). A skilled person will understand methods for determining suitable nucleotide substitutions based on codon options for each amino acid as they are known in the art.

Whilst preferable, codon optimised mRNA need not be uniformly codon optimised along the entire length of the mRNA molecule. Different nucleotide modifications and/or backbone structures may exist at various positions in the nucleic acid. One of ordinary skill in the art will appreciate that the modification(s) may be located at any position(s) of a mRNA such that the polypeptide function, polypeptide expression, mRNA thermal stability or structure is preferably increased or improved. A modification may also be a 5′ or 3′ terminal modification. Further, the mRNA may contain at a minimum one and at maximum 100% optimised codons, or any intervening percentage, such as at least 10%, at least 20%, at least 30%, at least 40%, at least 50%, at least 60%, at least 70%, at least 80%, or at least 90% optimised codons.

The % identity of a polynucleotide or mRNA is determined by GAP (Needleman and Wunsch, 1970) analysis (GCG program) with a gap creation penalty=5, and a gap extension penalty=0.3. The query sequence is at least 900 nucleotides in length, and the GAP analysis aligns the two sequences over a region of at least 900 nucleotides. Preferably, the query sequence is at least 975 nucleotides in length, and the GAP analysis aligns the two sequences over a region of at least 975 nucleotides. Even more preferably, the query sequence is at least 1,050 nucleotides in length and the GAP analysis aligns the two sequences over a region of at least 1,050 nucleotides. Even more preferably, the GAP analysis aligns two sequences over their entire length.

With regard to the defined polynucleotides or mRNA, it will be appreciated that % identity figures higher than those provided above will encompass preferred embodiments. Thus, where applicable, in light of the minimum % identity figures, it is preferred that the mRNA comprises a sequence which is at least 50%, at least 60%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9 identical to the relevant sequence.

When referring to polynucleotide or mRNA identity herein, it will be understood that a given sequence identity is in reference to the open reading frame sequence.

In a further embodiment, the present invention relates to polynucleotides or mRNA which are substantially identical or identical to those specifically described herein. As used herein, with reference to a mRNA or polynucleotide the term “substantially identical” means the substitution of one or a few (for example 2, 3, or 4) nucleotides whilst maintaining activity of the native protein encoded by the polynucleotide. In addition, this term includes the addition or deletion of nucleotides which results in the increase or decrease in size of the encoded native protein by one or a few (for example 2, 3, or 4) amino acids whilst maintaining activity of the native protein encoded by the polynucleotide.

### Chemical Modifications

The terms “modification” or “modified” refer to modification of a mRNA of the invention with respect to A, G, U or C ribonucleotides. Generally, the modification refers to the coding region of the mRNA. However, the modification may also be introduced into the flanking regions and/or the terminal regions if the modification increases protein expression, function, thermal stability or structure.

Because chemical bonds will necessarily be broken and reformed to effect a structural modification, structural modifications are of a chemical nature and hence are chemical modifications. However, structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

The mRNA or polynucleotides can include any useful modification, such as to the sugar, the nucleobase, or the internucleoside linkage (e.g. to a linking phosphate/to a phosphodiester linkage to the phosphodiester backbone). For example, the major groove of a polynucleotide, or the major groove face of a nucleobase may comprise one or more modifications. One or more atoms of a pyrimidine nucleobase (e.g. on the major groove face) may be replaced or substituted with optionally substituted amino, optionally substituted thiol, optionally substituted alkyl (e.g., methyl or ethyl), or halo (e.g., chloro or fluoro). In certain cases (e.g., one or more modifications) are present in each of the sugar and the internucleoside linkage. Modifications according to the present invention may be modifications of ribonucleic acids (RNAs) to deoxyribonucleic acids (DNAs), e.g., the substitution of the 2′OH of the ribofuranysyl ring to 2′H, threose nucleic acids (TNAs), glycol nucleic acids (GNAs), peptide nucleic acids (PNAs), locked nucleic acids (LNAs) or hybrids thereof). Additional modifications are known in the art.

In some embodiments the chemical modification increases mRNA stability and/or mRNA translation when compared to a mRNA without chemical modification. Preferably the modification is to a uracil (U) or a cytosine (C) but may be to any mRNA base including adenine (A), and guanine (G) if the modification increases mRNA stability and/or mRNA translation when compared to a mRNA without chemical modification.

Suitable uridine modifications may include but are not limited to pseudouridine (W), 5-aza-uridine, 6-aza-uridine, 2-thio-5-zauridine, 2-thio-uridine (s²U), 4-thio-uridine (s⁴U), 4-thiopseudouridine, 2-thio-pseudouridine, 5-hydroxy-uridine (ho5U), 5-aminoallyl-uridine, 5-halo-uridine (e.g., 5-iodo-uridine or 5-bromouridine), 3-methyluridine (m3U), 5-methoxyuridine (5moU), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyluridine (chm⁵U), 5-carboxyhydroxymethyl-uridine methyl ester (mchm5U), 5-20 methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm5U), 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyluridine, 1-propynyl-pseudouridine, 5-taurinomethyl-uridine (tm⁵U), 1-taurinomethyl-pseudouridine, 5-taurinomethyl-2-thio-uridine (tm⁵s²U), 1-taurinomethyl-4-thio-pseudouridine, 5-methyl-uridine (m⁵U, i.e., having the nucleobase deoxythymine), 1-methylpseudouridine (m¹ψ), 5-methyl-2-thiouridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-3 0 thio-1-methyl-1-deaza-pseudouridine, dihydrouridine (D), dihydropseudouridine, 5,6-dihydrouridine, 5-methyldihydrouridine (m⁵D), 2-thio-dihydrouridine, 2-thiodihydropseudouridine, 2-methoxy-uridine, 2-methoxy-4-thio-uridine, 4-methoxy-pseudouridine, 4-methoxy-2-thio-pseudouridine, N1-methyl-pseudouridine (also known as 1-methylpseudouridine (m¹ψ), 3-(3-amino-3-carboxypropyl)uridine (acp³U), 1-methyl-3-(3-amino-3-carboxypropyl)pseudouridine (acp³ψ), 5-(isopentenylaminomethyl)uridine (inm⁵U), 5-(isopentenylaminomethyl)-2-thio-uridine (inm⁵s²U), a-thio-uridine, 2′-O-methyl-uridine (Um), 5,2′-O-dimethyl-uridine (m5Um), 2′-O-methyl-pseudouridine (Win), 2-thio-2′-O-methyl-uridine (s2Um), 5-methoxycarbonylmethyl-2′-O-methyl-uridine (mcm5Um), 5-carbamoylmethyl-2′-Omethyl-uridine (ncm5Um), 5-carboxymethylaminomethyl-2′-O-methyluridine (cmnm5Um), 3,2′O-dimethyl-uridine (m3Um), 5-(isopentenylaminomethyl)-2′-O-methyl-uridine (inm5Um), 1-thio-5 uridine, deoxythymidine, 2′-F-arauridine, 2′-F-uridine, 2′-OH-ara-uridine, 5-(2-carbomethoxyvinyl) uridine, and 5-[3-(1-E-propenylamino)]uridine. Preferably, the uridine modification is 5-methoxyuridine (5moU) or N1-methylpseudouridine (m¹ψ), more preferably m¹ψ.

Suitable cytidine modifications may include but are not limited to 5-aza-cytidine, 6-aza-cytidine, pseudoisocytidine, 3-methyl-cytidine (m³C), N4-acetyl-cytidine (ac⁴C), 5-formyl-cytidine (PC), N4-methyl-cytidine (m⁴C), 5-methyl-cytidine (m⁵C), 5-methylcytidine (5mC), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thiozebularine, 2-methoxy-cytidine, 2-methoxy-5-methyl-cytidine, 4-methoxy-pseudoisocytidine, 4-methoxy-1-methyl-pseudoisocytidine, lysidine (k²C), 2-thiocytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴₂Cm), 1-thiocytidine, 2′-F-ara-cytidine, 2′-F-cytidine, and 2′-OH-ara-cytidine.

Modified nucleic acids need not be uniformly chemically modified along the entire length of the molecule. Different nucleotide modifications and/or backbone structures may exist at various positions in the nucleic acid. One of ordinary skill in the art will appreciate that the modification(s) may be located at any position(s) of a mRNA such that the polypeptide function, polypeptide expression, mRNA thermal stability or structure is preferably increased or improved. A modification may also be a 5′ or 3′ terminal modification. Further, the mRNA may contain at a minimum one and at maximum 100% chemical modifications, preferably nucleoside modifications, or any intervening percentage, such as at least 10%, at least 20%, at least 30%, at least 40%, at least 50%, at least 60%, at least 70%, at least 80%, or at least 90% modified nucleotides.

Methods of Synthesis of mRNA

mRNA for use in accordance with the invention may be prepared according to any available technique including, but not limited to chemical synthesis, enzymatic synthesis, which is generally termed in vitro transcription (IVT), enzymatic or chemical cleavage of a longer precursor, etc. Methods of synthesizing RNAs are known in the art.

In a preferred embodiment, a mRNA of the invention is prepared by IVT using methods described in the Example or known in the art. The machinery of the transfected cell is utilized for in vivo translation of the message to the corresponding protein, which is the therapeutic polypeptide suitable for treating a neurological disease or disorder described herein. IVT mRNA is engineered to structurally resemble naturally occurring mature and processed mRNA in the cytoplasm of eukaryotic cells. Hence, the IVT mRNA is single-stranded, has a 5′ cap and a 3′ poly(A) tail. The open reading frame (ORF) encoding the protein of interest is marked by start and stop codons and is flanked by untranslated region (UTRs). The mRNA is generally synthesized in a cell-free system by IVT from a DNA template, such as a linearized plasmid or a PCR product. With the exception of the 5′ cap, this DNA template encodes all the structural elements of a functional mRNA. IVT is performed with T7 or SP6 RNA polymerase in the presence of nucleotides and thereafter the mRNA is capped enzymatically. The template DNA is then digested by DNAses and the mRNA is purified by conventionally used methods for isolating nucleic acids.

### Nucleic Acids and Vectors

The present invention relates to various polynucleotides preferably encoding mRNA, particularly those used in IVT for the generation of a mRNA of the invention. As used herein, a “polynucleotide” or “nucleic acid” or “nucleic acid molecule” means a polymer of nucleotides, which may be DNA or RNA or a combination thereof, and includes genomic DNA, mRNA, cRNA, and cDNA. A given polynucleotide may be of cellular, genomic or synthetic origin, for example made on an automated synthesizer, and may be combined with carbohydrate, lipids, protein or other materials, labelled with fluorescent or other groups, or attached to a solid support to perform a particular activity defined herein, or comprise one or more modified nucleotides not found in nature, well known to those skilled in the art. The polymer may be single-stranded, essentially double-stranded or partly double-stranded. Basepairing as used herein refers to standard basepairing between nucleotides, including G:U basepairs. “Complementary” means two polynucleotides are capable of basepairing (hybridizing) along part of their lengths, or along the full length of one or both. The term “polynucleotide” is used interchangeably herein with the term “nucleic acid”.

The polynucleotides or nucleic acid sequences of the present application may be deoxyribonucleic acid (DNA) sequences or ribonucleic acid (RNA) sequences and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The nucleic acid can be either double stranded or single stranded, and represents the sense or antisense strand. Further, the term “nucleic acid” includes the complementary nucleic acid sequences.

The term “nucleic acid molecule” or its derivatives, as used herein, is intended to include unmodified DNA or RNA or modified DNA or RNA For example, it may be useful for the nucleic acid molecules of the disclosure to be composed of single- and double-stranded DNA, DNA that is a mixture of single- and double-stranded regions, single- and double-stranded RNA, and RNA that is a mixture of single- and double stranded regions, hybrid molecules comprising DNA and RNA that may be single stranded or, more typically double-stranded or a mixture of single- and double-stranded regions. In addition, it may be useful for the nucleic acid molecules to be composed of triple stranded regions comprising RNA or DNA or both RNA and DNA The nucleic acid molecules of the disclosure may also contain one or more modified bases or DNA or RNA backbones modified for stability or for other reasons. “Modified” bases include, for example, tritiated bases and unusual bases such as inosine. A variety of modifications can be made to DNA and RNA; thus “nucleic acid molecule” encompasses chemically, enzymatically, or metabolically modified forms. The term “polynucleotide” shall have a corresponding meaning.

The term “isolated polynucleotide” means a polynucleotide which has generally been separated from the polynucleotide sequences with which it is associated or linked in its native state if the polynucleotide is found in nature. Preferably, the isolated polynucleotide is at least 90% free from other components with which it is naturally associated, if it is found in nature. Preferably the polynucleotide is not naturally occurring, for example by covalently joining two shorter polynucleotide sequences in a manner not found in nature (chimeric polynucleotide).

A genomic form or clone of a gene containing the transcribed region may be interrupted with non-coding sequences termed “introns” or “intervening regions” or “intervening sequences” which may be either homologous or heterologous with respect to the “exons” of the gene. An “intron” as used herein is a segment of a gene which is transcribed as part of a primary RNA transcript but is not present in the mature mRNA molecule. Introns are removed or “spliced out” from the nuclear or primary transcript; introns therefore are absent in the messenger RNA (mRNA). Introns may contain regulatory elements such as enhancers. “Exons” as used herein refer to the DNA regions corresponding to the RNA sequences which are present in the mature mRNA or the mature RNA molecule in cases where the RNA molecule is not translated. An mRNA functions during translation to specify the sequence or order of amino acids in a nascent polypeptide. The term “gene” includes a synthetic or fusion molecule encoding all or part of the proteins of the invention described herein and a complementary nucleotide sequence to any one of the above.

As used herein, a “chimeric gene” refers to any gene that comprises covalently joined sequences that are not found joined in nature. Typically, a chimeric gene comprises regulatory and transcribed or protein coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. The term “endogenous” is used herein to refer to a mRNA that is normally present in a mammalian cell and refers to a native gene in its natural location in the genome of an organism. It may also be referred to as wild-type. As used herein, “recombinant nucleic acid molecule”, “recombinant polynucleotide” or variations thereof refer to a nucleic acid molecule which has been constructed or modified by recombinant DNA/RNA technology. The terms “foreign polynucleotide” or “exogenous polynucleotide” or “heterologous polynucleotide” and the like refer to any nucleic acid which is introduced into the genome of a cell by experimental manipulations.

Foreign or exogenous genes may be genes that are inserted into a non-native organism or cell, native genes introduced into a new location within the native host, or chimeric genes. Alternatively, foreign or exogenous genes may be the result of editing the genome of the organism or cell, or progeny derived therefrom. A “transgene” is a gene that has been introduced into the genome by a transformation procedure.

The term “genetically engineered”, “genetically modified”, “genetic modification” or variants thereof refers to any genetic manipulation by man and includes introducing genes into cells by transformation or transduction, gene editing, cisgenesis, mutating genes in cells and altering or modulating the regulation of a gene in a cell or organisms to which these acts have been done or their progeny and so on.

Furthermore, the term “exogenous” in the context of a polynucleotide (nucleic acid) refers to the polynucleotide when present in a cell that does not naturally comprise the polynucleotide. The cell may be a cell which comprises a non-endogenous polynucleotide resulting in an altered amount of production of the encoded polypeptide, for example an exogenous polynucleotide which increases the expression of an endogenous polypeptide, or a cell which in its native state does not produce the polypeptide. Increased production of a polypeptide is also referred to as “over-expression”. An exogenous polynucleotide of the invention includes polynucleotides which have not been separated from other components of the transgenic (recombinant) cell, or cell-free expression system, in which it is present, and polynucleotides produced in such cells or cell-free systems which are subsequently purified away from at least some other components. The exogenous polynucleotide (nucleic acid) can be a contiguous stretch of nucleotides existing in nature, or comprise two or more contiguous stretches of nucleotides from different sources (naturally occurring and/or synthetic) joined to form a single polynucleotide. Typically, such chimeric polynucleotides comprise at least an open reading frame encoding a polypeptide of the invention operably linked to a promoter suitable of driving transcription of the open reading frame in a cell of interest.

The present invention also relates to the use of oligonucleotides, for instance in methods of screening for a mRNA molecule of the invention. As used herein, “oligonucleotides” are polynucleotides up to 50 nucleotides in length. The minimum size of such oligonucleotides is the size required for the formation of a stable hybrid between an oligonucleotide and a complementary sequence on a nucleic acid molecule of the present invention. They can be RNA, DNA, or combinations or derivatives of either. Oligonucleotides are typically relatively short single stranded molecules of 10 to 30 nucleotides, commonly 15-25 nucleotides in length. When used as a guide for genome editing, probe or as a primer in an amplification reaction, the minimum size of such an oligonucleotide is the size required for the formation of a stable hybrid between the oligonucleotide and a complementary sequence on a target nucleic acid molecule. Preferably, the oligonucleotides are at least 15 nucleotides, more preferably at least 18 nucleotides, more preferably at least 19 nucleotides, more preferably at least 20 nucleotides, more preferably at least 22 nucleotides, even more preferably at least 25 nucleotides in length. Oligonucleotides of the present invention used as a probe are typically conjugated with a label such as a radioisotope, an enzyme, biotin, a fluorescent molecule or a chemiluminescent molecule.

As those skilled in the art would be aware, the sequence of the oligonucleotide primers described herein can be varied to some degree without affecting their usefulness for the methods of the invention. A “variant” of an oligonucleotide disclosed herein (also referred to herein as a “primer” or “probe” depending on its use) useful for the methods of the invention includes molecules of varying sizes of, and/or are capable of hybridising to the genome close to that of, the specific oligonucleotide molecules defined herein. For example, variants may comprise additional nucleotides (such as 1, 2, 3, 4, or more), or less nucleotides as long as they still hybridise to the target region. Furthermore, a few nucleotides may be substituted without influencing the ability of the oligonucleotide to hybridise the target region. In addition, variants may readily be designed which hybridise close (for example, but not limited to, within 50 nucleotides or within 100 nucleotides) to the region of the genome where the specific oligonucleotides defined herein hybridise.

The present invention includes oligonucleotides that can be used as, for example, guides for RNA-guided endonucleases, probes to identify nucleic acid molecules, or primers to produce nucleic acid molecules. Probes and/or primers can be used to clone homologues of the polynucleotides of the invention from other species. Furthermore, hybridization techniques known in the art can also be used to screen genomic or cDNA libraries for such homologues.

Polynucleotides and oligonucleotides of the present invention include those which hybridize under stringent conditions to one or more of the sequences disclosed herein. As used herein, stringent conditions are those that (1) employ low ionic strength and high temperature for washing, for example, 0.015 M NaCl/0.0015 M sodium citrate/0.1% NaDodSQ4 at 50° C.; (2) employ during hybridisation a denaturing agent such as formamide, for example, 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 g/ml), 0.1% SDS and 10% dextran sulfate at 42° C. in 0.2×SSC and 0.1% SDS.

Polynucleotides of the present invention may possess, when compared to naturally occurring molecules, one or more mutations which are deletions, insertions, or substitutions of nucleotide residues. Mutants can be either naturally occurring (that is to say, isolated from a natural source) or synthetic (for example, by performing site-directed mutagenesis on the nucleic acid). A variant of a polynucleotide of the invention includes molecules of varying sizes when compared to the reference polynucleotides defined herein. For example, variants may comprise additional nucleotides (such as 1, 2, 3, 4, or more), or less nucleotides as long as they encode a functional protein. Furthermore, a few nucleotides may be substituted without influencing the integrity of the encoded protein. In addition, variants may include polynucleotides which encode the same polypeptide or amino acid sequence but which vary in nucleotide sequence by redundancy of the genetic code. The terms “polynucleotide variant” and “variant” also include naturally occurring allelic variants.

In an aspect of the invention, CD98hc antibodies used in accordance with the invention may be expressed from a nucleic acid encoding a CD98hc antibody, preferably a CD98hc antibody. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., YO, NS0, Sp20 cell). In one embodiment, a method of making an antiCD98hc antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-CD98hc antibody, a nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fe effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248, pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gemgross, Nat. Biotech. 22: 1409-1414 (2004), and Li et al., Nat. Biotech. 24:210-215 (2006).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J Gen Viral. 36:59 (1977)); baby hamster kidney cells (BHK); mouse sertoli cells (TM4 cells as described, e.g., in Mather, Biol. Reprod 23:243-251 (1980); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals NY Acad Sci. 383:44-68 (1982); MRC 5 cells; and FS4 cells. Other useful mammalian host cell lines include Chinese hamster ovary (CHO) cells, including DHFR″ CHO cells (Urlaub et al., Proc. Natl. Acad Sci. USA 77:4216 (1980)); and myeloma cell lines such as YO, NS0 and Sp2/0.

### Methods for Detection

In some aspects, methods of detecting CD98hc on the blood-brain barrier are provided. Thus, in some aspects, an anti-CD98hc antibody binds to CD98hc with sufficient affinity such that the antibody is useful as a detection agent in targeting CD98hc. In certain embodiments, the anti-CD98hc antibody is useful for detecting the presence of CD98hc in a biological sample. The term “detecting” as used herein encompasses quantitative or qualitative detection. In certain embodiments, a biological sample comprises a cell or tissue, such as brain cells, e.g., brain capillary endothelial cells.

By way of example, an antibody disclosed herein may be conjugated to an imaging agent. Following administration of the antibody conjugate, the imaging agent may be detected, e.g., in isolated brain tissue, and/or using in vivo brain imaging techniques (e.g., using bioluminescence or fluorescence) (see, e.g., J. R. Martin. J Neurogenet. 2008; 22(3):285-307). Suitable labels for in vitro or in vivo use include labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes 32P, 14C, 1251, 3H, and 1311, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, ˜-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

In one embodiment, an anti-CD98hc antibody for use in a method of detection is provided. In a further aspect, a method of detecting the presence of CD98hc in a biological sample is provided. In certain embodiments, the method comprises contacting the biological sample with an anti-CD98hc antibody as described herein under conditions permissive for binding of the anti-CD98hc antibody to CD98hc, and detecting whether a complex is formed between the anti-CD98hc antibody and CD98hc.

### Cells

In an aspect of the invention, there is provided a mammalian cell or a host cell comprising a nucleic acid of the invention. In an embodiment, the host cell is a neuron or a glial cell, wherein the glial cell is selected from the group consisting of astrocytes, oligodendrocytes, ependymal cells, and microglia. As it is however envisaged that the LNP or compositions or the invention are suitable for expression in the liver and spleen the host or mammalian cell may be a hepatocyte, reticular cell, lymphocyte or epithelial cell.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cells and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. Examples of “host cells” for producing recombinant antibodies include: (1) mammalian cells, for example, Chinese Hamster Ovary (CHO), COS, myeloma cells (including YO and NS0 cells), baby hamster kidney (BHK), Hela and Vero cells; (2) insect cells, for example, sf9, sf21 and Tn5; (3) yeast cells, for example, those belonging to the genus Saccharomyces (e.g. Saccharomyces cerevisiae) or the genus Aspergillus (e.g. Aspergillus niger); (5) bacterial cells, for example Escherichia coli cells or Bacillus subtilis cells, etc

The host cell is preferably a human cell but may be any mammalian cell including those from pets (i.e., cats, rabbits, guinea pigs, ferrets, dogs) or livestock animals (cattle, pigs, sheep, goats, chickens, camelids, deer, bison, buffalo and related species including wild and zoo animals). In an embodiment, the host cell or mammalian cell is non-human.

### Pharmaceutical Compositions

In an aspect of the invention, there is provided a pharmaceutical composition comprising a pharmaceutically acceptable carrier, diluent or excipient. In some examples, a pharmaceutical composition described herein can be administered orally, parenterally, by inhalation spray, adsorption, absorption, topically, rectally, nasally, bucally, intraventricularly, via an implanted reservoir in dosage formulations containing conventional non-toxic pharmaceutically-acceptable carriers, or by any other convenient dosage form. The term “parenteral” as used herein includes subcutaneous, intravenous, intramuscular, intraperitoneal, intrathecal, intraventricular, intrasternal, and intracranial injection or infusion techniques. Because the present invention provides the advantage of avoiding surgical administration of a pharmaceutical composition, it is envisaged that the pharmaceutical compositions are most suitable for intravenous administration.

Methods for preparing a LNP into a suitable form for administration to a subject (e.g. a pharmaceutical composition) are known in the art and include, for example, methods as described in Remington and Gennaro (1990) and U.S. Pharmacopeia: National Formulary (1984).

The pharmaceutical compositions of this invention are useful for parenteral administration, such as intravenous administration or administration into a body cavity or lumen of an organ or joint. The compositions for administration will commonly comprise a suspension of LNPs suspended in a pharmaceutically acceptable carrier, for example an aqueous carrier. A variety of aqueous carriers can be used, e.g., PBS, Tris buffer, and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of a nucleic acid of the present invention in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives.

Pharmaceutically acceptable acidic/anionic salts for use in the invention include and are not limited to acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate and triethiodide. Organic or inorganic acids also include, and are not limited to, hydriodic, perchloric, sulfuric, phosphoric, propionic, glycolic, methanesulfonic, hydroxyethanesulfonic, oxalic, 2-naphthalenesulfonic, p-toluenesulfonic, cyclohexanesulfamic, saccharinic or trifluoroacetic acid. Pharmaceutically acceptable basic/cationic salts include, and are not limited to aluminum, 2-amino-2-hydroxymethyl-propane-1,3-diol (also known as tris(hydroxymethyl)aminomethane, tromethane or “TRIS”), ammonia, benzathine, t-butylamine, calcium, chloroprocaine, choline, cyclohexylamine, diethanolamine, ethylenediamine, lithium, lysine, magnesium, meglumine, N-methyl-D-glucamine, piperidine, potassium, procaine, quinine, sodium, triethanolamine, or zinc.

In less preferred embodiments, the compositions of the present invention are administered intracranially, for instance injected into the brain, such as by direct injection into the brain. Direct injection may be performed by intraventricular and intracerebral routes. Injection of the compositions into the brain can also be performed using a device for administration. Direct administration of the drugs into the central nervous system may also be achieved by using epidural (injection or infusion into the epidural space), intracerebral (into the cerebrum), intracerebroventricular (into the cerebral ventricles), or intrathecal (into the spinal canal) injection. Pathan et al. (2009), incorporated by reference in its entirety herein, describes some methods of administration of a composition to the brain and others are known in the art. Alternatively, the compositions of the present invention may be administered to the central nervous system by systemic delivery, preferably by intravenous delivery.

In certain embodiments, the target site can be located in any region of the CNS, including the brain and the spinal cord. A site of administration within the CNS can be chosen based on the desired target region of neuropathology and, optionally, the topology of brain circuits involved when an administration site and the target region have axonal connections. In certain embodiments, the target region can be defined, for example, using 3-D stereotaxic coordinates. An administration site may be localized in a region innervated by projection neurons connecting distal regions of the brain. For example, the substantia nigra and ventral tegmental area send dense projections to the caudate and putamen (collectively known as the striatum). Neurons within the substantia nigra and ventral tegmentum can be targeted for transduction by retrograde transport of a composition following injection into the striatum. As another example, the hippocampus receives well-defined, predictable axonal projections from other regions of the brain. Other administration sites may be localized, for example, in the spinal cord, brainstem (medulla and pons), mesencephalon, cerebellum, diencephalon (thalamus, hypothalamus), telencephalon (corpus striatum, cerebral cortex, or, within the cortex, the occipital, temporal, parietal or frontal lobes), or combinations thereof.

Although less preferable, to deliver a pharmaceutical composition described herein specifically to a particular region of the central nervous system, especially to a particular region of the brain, it may be administered by stereotaxic microinjection. For example, on the day of surgery, patients will have the stereotaxic frame base fixed in place (screwed into the skull). The brain with stereotaxic frame base (MM-compatible with fiduciary markings) will be imaged using high resolution MM. The MM images will then be transferred to a computer that runs stereotaxic software. A series of coronal, sagittal and axial images will be used to determine the target site of vector injection, and trajectory. The software directly translates the trajectory into 3-dimensional coordinates appropriate for the stereotaxic frame. Burr holes are drilled above the entry site and the stereotaxic apparatus localized with the needle implanted at the given depth. The pharmaceutical composition will then be administrated by direct injection to the primary target site and retrogradely transported to distal target sites via axons. Additional routes of administration may be used, e.g., intrathecal injection, superficial cortical application under direct visualization, or other non-stereotaxic application.

In another aspect, the invention provides a method of delivering a pharmaceutical composition to a target cell of the CNS, which is a neuron or a glial cell, in a subject with a neurological disease or disorder. The method comprises contacting an axonal ending of a neuron with a pharmaceutical composition; allowing the pharmaceutical composition to be endocytosed and retrogradely transported intracellularly along the axon to the nucleus of the neuron; allowing the polypeptide to be expressed and transported within the membrane(s) of the neuron, wherein the polypeptide is therefore capable of treating a desired neurological disease or disorder.

Upon formulation, a pharmaceutical composition of the present invention will be administered in a manner compatible with the dosage formulation and in such amount as is therapeutically effective. Formulations are easily administered in a variety of dosage forms, such as the type of injectable solutions described above, but other pharmaceutically acceptable forms are also contemplated, e.g., tablets, pills, capsules or other solids for oral administration, suppositories, pessaries, nasal solutions or sprays, aerosols, inhalants, LNP forms and the like. Pharmaceutical “slow release” capsules or compositions may also be used. Slow-release formulations are generally designed to give a constant drug level over an extended period and may be used to deliver a LNP of the present invention.

Suitable dosages of a pharmaceutical composition of the present invention will vary depending on the specific nucleic acid used and/or the subject being treated. It is within the ability of a skilled physician to determine a suitable dosage, e.g., by commencing with a sub-optimal dosage and incrementally modifying the dosage to determine an optimal or useful dosage. Alternatively, to determine an appropriate dosage for treatment/prophylaxis, data from the cell culture assays or animal studies are used, wherein a suitable dose is within a range of circulating concentrations that include the ED50 of the active compound with little or no toxicity. The dosage may vary within this range depending upon the dosage form employed and the route of administration utilized. A therapeutically effective dose can be estimated initially from cell culture assays. A dose may be formulated in animal models to achieve a circulating plasma concentration range that includes the IC50 (i.e., the concentration or amount of the compound which achieves a half-maximal inhibition of symptoms) as determined in cell culture. Such information can be used to more accurately determine useful doses in humans. Levels in plasma may be measured, for example, by high performance liquid chromatography.

In some examples, a method of the present invention comprises administering a therapeutically effective amount of a pharmaceutical composition described herein. In this context, the therapeutically effective amount refers to the amount of pharmaceutical composition provided to a target cell or tissue required to exert a therapeutic effect in the treatment of a neurological disease or disorder.

The term “therapeutically effective amount” is the quantity which, when administered to a subject in need of treatment, improves the prognosis and/or state of the subject and/or that reduces or inhibits one or more symptoms of a clinical condition described herein to a level that is below that observed and accepted as clinically diagnostic or clinically characteristic of that condition. The amount to be administered to a subject will depend on the particular characteristics of the condition to be treated, the type and stage of condition being treated, the mode of administration, and the characteristics of the subject, such as general health, other diseases, age, sex, genotype, and body weight. A person skilled in the art will be able to determine appropriate dosages depending on these and other factors. Accordingly, this term is not to be construed to limit the present invention to a specific quantity, e.g., weight or amount of protein(s), rather the present invention encompasses any amount of the nucleic acid sufficient to achieve the stated result in a subject.

The present invention provides for both prophylactic and therapeutic methods of treating a subject at risk of (or susceptible to) a neurological disease or disorder via tissue specific or systemic delivery of a pharmaceutical composition described herein.

The term “prophylactically effective amount” shall be taken to mean a sufficient quantity of a pharmaceutical composition that prevents or inhibits or delays the onset of one or more detectable symptoms of a clinical condition. The skilled person will be aware that such an amount will vary depending on, for example, the specific nucleic acid conjugated to the LNP administered and/or the particular subject and/or the type or severity or level of condition and/or predisposition (genetic or otherwise) to the condition. Accordingly, this term is not to be construed to limit the present invention to a specific quantity, e.g., weight or amount of nucleic acid or a pharmaceutical composition thereof, rather the present invention encompasses any amount of a pharmaceutical composition thereof sufficient to achieve the stated result in a subject.

As used herein, a subject “at risk” of developing a neurological disease or disorder may have or may not have detectable symptoms of a lysosomal storage disease, and may have or may not have displayed detectable symptoms of a neurological disease or disorder prior to a treatment according to the present disclosure. “At risk” denotes that a subject has one or more risk factors, which are measurable parameters that correlate with development of a neurological disease or disorder, as known in the art and/or described herein.

As used herein, “preventing” or “prevention” is intended to refer to at least the reduction of likelihood of the risk of (or susceptibility to) acquiring a disease or disorder (i.e., causing at least one of the clinical symptoms of the disease not to develop in a patient that may be exposed to or predisposed to the disease but does not yet experience or display symptoms of the disease).

Biological and physiological parameters for identifying such patients are provided herein and are also well known by physicians. In relation to the present disclosure, prevention of neurological disease or disorder may be achieved where a subject does not have any clinical symptoms of the disease or disorder and may be considered asymptomatic.

### Methods of Treatment

Provided herein are methods of treating or preventing a neurological disease or disorder in a subject in need thereof, comprising administering a composition of the invention to the subject, thereby treating or preventing the neurological disease or disorder in the subject.

A “neurological disorder” as used herein refers to a disease or disorder which affects the CNS and/or which has an etiology in the CNS. The CNS will be understood to include the eye, which is normally sequestered from the rest of the body by the blood-retina barrier.

In some embodiments, the method further comprises administering to the subject an effective amount of at least one additional therapeutic agent. Such additional treatments which may be administered together or separately.

In certain embodiments, an additional therapeutic agent is a therapeutic agent effective to treat the same or a different neurological disorder as the anti-CD98hc antibody or antigen binding fragment or conjugate thereof is being employed to treat. In certain embodiments, the at least one additional therapeutic agent is selected for its ability to mitigate one or more side effects of the neurological drug. The additional therapeutic agent can be administered in the same or separate formulations and administered together or separately with the anti-CD98hc antibody or antigen binding fragment or conjugate thereof. The anti-CD98hc antibody or antigen binding fragment or conjugate of the application can be administered prior to, simultaneously with, and/or following, the administration of the additional therapeutic agent and/or adjuvant. The anti-CD98hc antibody or antigen binding fragment or conjugate thereof of the application can also be used in combination with other interventional therapies such as, but not limited to, radiation therapy, behavioral therapy, or other therapies known in the art and appropriate for the neurological disorder to be treated or prevented.

A subject in need of treatment may present a number of symptoms depending on the type of disorder that is present. In an aspect, a subject in need of treatment may exhibit well characterised symptoms associated with a disease or condition described herein.

The terms “treatment” or “treating” of a subject includes the administration of a LNP or pharmaceutical composition of the invention to a subject with the purpose of delaying, slowing, stabilizing, curing, healing, alleviating, relieving, altering, remedying, less worsening, ameliorating, improving, or affecting the condition or disease, or a symptom of the disease or condition including those listed above.

The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury. In particular, treating refers to a reduction in a symptoms associated with the disease or condition. Any improvement may be determined directly from the subject, or a sample or biopsy therefrom. The sample or biopsy may be of the diseased tissue.

### Kits

In an aspect, the present invention provides a kit comprising one or more of the following:
(i) a nucleic acid of the invention;
(ii) a pharmaceutical composition of the invention.

In the case of a kit for therapeutic use, the kit can additionally comprise a pharmaceutically acceptable carrier, diluent or excipient.

Optionally, a kit of the invention is packaged with instructions for use in a method described herein according to any example.

In another aspect, the application relates to an article of manufacture (such as a kit) containing materials useful for the treatment, prevention and/or diagnosis of the neurological diseases or disorders described above is provided. The article of manufacture comprises a container and a label or package insert on or associated with the container. Suitable containers include, for example, bottles, vials, syringes, IV solution bags, etc. The containers can be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper plerceable by a hypodermic injection needle). At least one active agent in the composition is a nucleic acid. The label or package insert indicates that the composition is used for treating the condition of choice. Moreover, the article of manufacture can include (a) a first container with a composition contained therein, wherein the composition comprises an antibody, antigen binding fragment thereof or a conjugate of the application; and (b) a second container with a composition contained therein, wherein the composition comprises a further therapeutic agent. The article of manufacture in this embodiment of the invention can further include a package insert indicating that the compositions can be used to treat a particular condition. Optionally, the article of manufacture can further comprise a second (or third) container comprising a pharmaceutically acceptable buffer, such as bacteriostatic water for injection, phosphate-buffered saline, Ringer's solution and dextrose solution. It can further include other materials desirable from a commercial and user standpoint, including other buffers, diluents, filters, needles, and syringes.

### EXAMPLES

### Example 1

This Example was conducted to determine whether the LNP compositions described herein could successfully provide for a non-invasive approach to deliver mRNA-based therapeutics through the BBB to the central nervous system.

### Materials and Methods

Reagents and materials. Phosphate-buffered saline (PBS) and UltraPure Dnase/Rnase-Free Distilled Water were purchased from Invitrogen. DOPE, DSPC, cholesterol, and DMG-PEG 2000 were purchased from Avanti Polar Lipids. Fluorescent lipophilic tracers DiD and DiR were purchased from Invitrogen. SM-102, ALC-0315 and β-sitosterol were purchased from APExBIO. 1,2-distearoyl-sn-glycero-3-phosphoethanolamine-N-[maleimide(polyethylene glycol)-2000](ammonium salt) (DSPE-PEG(2000) Maleimide) was purchased from Biopharma PEG. DLin-MC3-DMA and DOTAP were purchased from MedChemExpress. Monoclonal IgG antibodies targeted against CD98hc were purchased from BioLegend (clone RL388), Bio-Rad (clone H202-529.4.1.4) and Miltenyi Biotec (biotinylated; clone REA861). A monoclonal IgG antibody targeting CD3ε was purchased from BD Biosciences (clone 145-2C11). Primary rabbit monoclonal antibodies against NeuN (clone D4G40), glial fibrillary acidic protein (GFAP; clone E4L7M), allograft inflammatory factor 1 (AIF-1; clone E4O4W), and myelin basic protein (MBP; clone D8X4Q) were purchased from Cell Signaling Technologies. An Alexa Fluor® 488-labeled primary rat monoclonal antibody against CD45R (clone RA3-6B2) and an Alexa Fluor® 555-labeled primary hamster monoclonal antibody against CD3ε (clone 145-2C11) were purchased from BD Biosciences. Alexa Fluor 488- and 555-labeled anti-rabbit secondary antibodies were purchased from Cell Signaling Technologies. Lectin DyLight 488 was purchased from Sigma-Aldrich.

mRNA production. All mRNA constructs used in this study were produced via in vitro transcription (IVT) as per established protocols. Briefly, coding regions for each construct were codon optimised and flanked by highly expressing 5′ and 3′ untranslated regions, as well as a 100-nucleotide poly A tail at the 3′ end. All mRNA constructs were co-transcriptionally capped with a Cap1 structure and base modified via complete substitution of uridine triphosphate with N1-methylpseudouridine-5′-triphosphate (APExBIO) to ensure maximal translatability and minimal immunogenicity of the generated constructs. All mRNAs were rigorously purified to ensure that no IVT reaction contaminants, including immunogenic dsRNA byproducts, were present in the final formulations.

Lipid nanoparticle formulation. LNPs were formulated using a microfluidic mixing technique, following a previously described method.³⁸ In brief, an ethanol phase was prepared comprising an ionizable lipid, phospholipid, sterol, and PEG-lipid, at a molar ratio of 50:10:38.5:1.5. To enable direct labeling of LNPs for in vivo biodistribution experiments, at times a lipid fluorescent dye (DiR or DiD) was added to the ethanol phase (ionizable lipid/phospholipid/sterol/PEG-lipid/dye molar ratio of 50/10/38.4/1.5/0.1). Using a NanoAssemblr Ignite (Precision Nanosystems), this ethanol phase was rapidly mixed with an aqueous solution of mRNA in 20 mM citrate buffer, pH 4.0, at an aqueous-to-ethanol volume ratio of 3:1 and a combined flowrate of 12 mL/min. LNPs were subsequently dialyzed against PBS overnight at 4° C., using Pur-A-Lyzer Maxi 6000 Dialysis Kits (Sigma). Nanoparticle size and polydispersity index were measured by dynamic light scattering using a Malvern Zetasizer (Malvern Instruments). Final mRNA concentrations and encapsulation efficiencies were measured using a modified Quant-iT RiboGreen RNA assay (Invitrogen), as per established protocols (McKenzie et al., Current Protocols 3(9): e898 (2023)).

Antibody conjugation. LNPs were conjugated with monoclonal antibodies specific for either CD98hc or CD3. Briefly, antibodies were labelled with an excess amount of N-succinimidyl S-acetylthioacetate (SATA; SATA:antibody molar ratio of 40:1) at room temperature for 30 minutes. Unreacted SATA was removed using Zeba™ spin desalting columns (7 kD cutoff, Thermo Fisher Scientific). SATA-labelled antibodies were deprotected with deacetylation buffer (0.5 M hydroxylamine, 25 mM EDTA in PBS) at a SATA-antibody:buffer volumetric ratio of 10:1. The hydroxylamine was removed using Zeba™ spin desalting columns. For conjugation to LNPs, 0.5 mol % DSPE-PEG-maleimide was post-inserted into assembled LNPs via co-incubation for 30 minutes at 40° C. The term “post-inserted” will be understood to mean that the 0.5 mol % DSPE-PEG-maleimide was inserted into the LNP after the LNP was formed. Maleimide-activated LNPs were reacted with deprotected SATA-labelled antibodies at room temperature overnight. Antibody-conjugated LNPs were either directly administered via intravenous injection into mice or purified prior to administration via size exclusion chromatography using a Sephacryl S-300 High Resolution (Cytiva) packed column. Labelled LNPs were analyzed for size, polydispersity and ζ-potential using a Malvern Zetasizer, and concentration using a Nanosight NS300 (Malvern Panalytical). Antibody-conjugated LNP formulations were stored at 4° C. and used within 5 days of preparation.

Bioluminescence imaging. C57BL/6J mice 8-10 weeks of age and weighing between 15-20 g were administered saline solution or varying dosages of different mRNA-LNP formulations via lateral tail vein injection (n=3-4 per treatment condition). At the stated timepoints, mice were euthanized with CO₂. Major organs, including the brain, liver, spleen, kidney, heart, lungs, and lymph nodes, were harvested immediately after euthanasia. For fluorescent reporter mRNA formulations (e.g., mFlame™ mRNA), organs were immediately imaged using an IVIS Lumina system (PerkinElmer). For Fluc mRNA formulations, organs were immersed in a solution of 0.3 mg/mL IVISbrite D-Luciferin Bioluminescent Substrate (Revvity) for up to 15-30 minutes, and then subjected to IVIS imaging.

Immunofluorescence microscopy. Following intravenous injection of saline solution or various mRNA-LNP formulations, mice were euthanized via CO₂ inhalation at the stated timepoints. Key organs were harvested, fixed in 4% PFA (diluted in 1×PBS) at 4° C. overnight, and dehydrated via a two-step immersion process, first in 15% sucrose solution (diluted in 1×PBS) at 4° C. overnight, and then in 30% sucrose solution (diluted in 1×PBS) at 4° C. overnight. Dehydrated tissues were embedded in frozen optimal cutting temperature (OCT) medium and stored at −80° C. until required for sectioning. Sectioning was conducted on a Leica Biosystems CM1950 Cryostat (Leica Biosystems) and tissue sections were deposited on glass microscope slides. For immunostaining, tissue sections were first immersed in Blocking Buffer (5% BSA and 0.3% Triton™ X-100 in 1×PBS) for one hour at room temperature, and subsequently incubated at 4° C. overnight with primary antibodies diluted in Antibody Dilution Buffer (1% BSA and 0.% Triton™ X-100 in 1×PBS) as per the supplier's recommendation. After washing three times in 1×PBS, specimens were incubated for two hours at room temperature in the dark with the relevant species-specific fluorochrome-conjugated secondary antibody diluted as per the supplier's recommendation in Antibody Dilution Buffer. Samples were washed three times with 1×PBS, stained with DAPI (Vector Laboratories), mounted with a glass coverslip, and allowed to cure overnight at room temperature in the dark. Tissue sections were imaged and analyzed either via confocal laser-scanning microscopy using a Nikon A1R+ Confocal Microscope (Nikon) or via whole slide fluorescence imaging using an Axioscan 7 Microscope Slide Scanner (Zeiss).

Animal husbandry. All in vivo experiments conducted as part of this work were carried out in accordance with the Australian code for the care and use of animals for scientific purposes. Experimental protocols were approved by the University of Melbourne Animal Ethics Committee (Ethics ID 2023-10404-41343-17). All mice were sourced from the Melbourne Bioresources Platform. Mice were housed on a 12 h light/dark cycle and were provided with ad libitum access to food and water.

Statistics. The number of mice used in each in vivo experiment is indicated in individual figure legends. Experiments were not randomized, and no statistical analyses were conducted to predetermine sample sizes. Study data were analyzed using GraphPad Prism 9 (GraphPad), by either ordinary one-way ANOVA with a Dunnett's multiple comparisons test or two-way ANOVA with a Tukey's multiple comparisons test Data are expressed as mean SD. ****P<0.0001; ***P<0.001; **P<0.01; *P<0.05; NS, P>0.05.

### Results

CD98hc-Targeted LNPs Enable Delivery of Diverse mRNA Cargoes into the Mouse Brain after Intravenous Injection

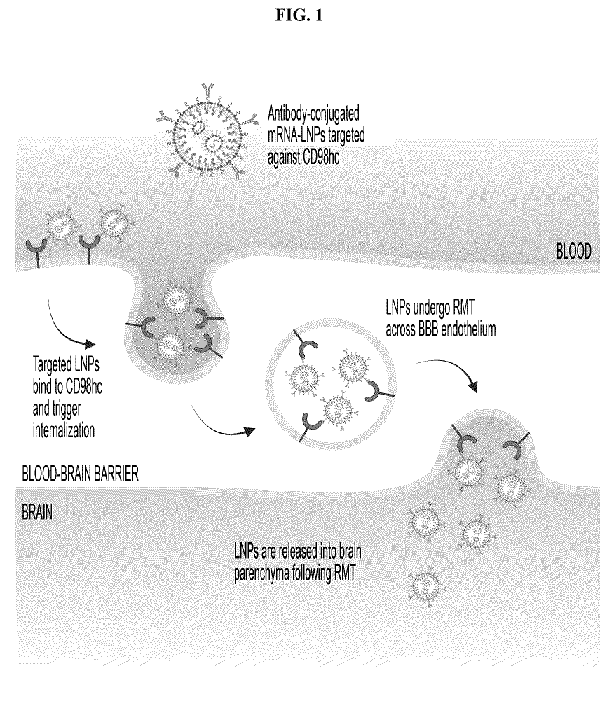

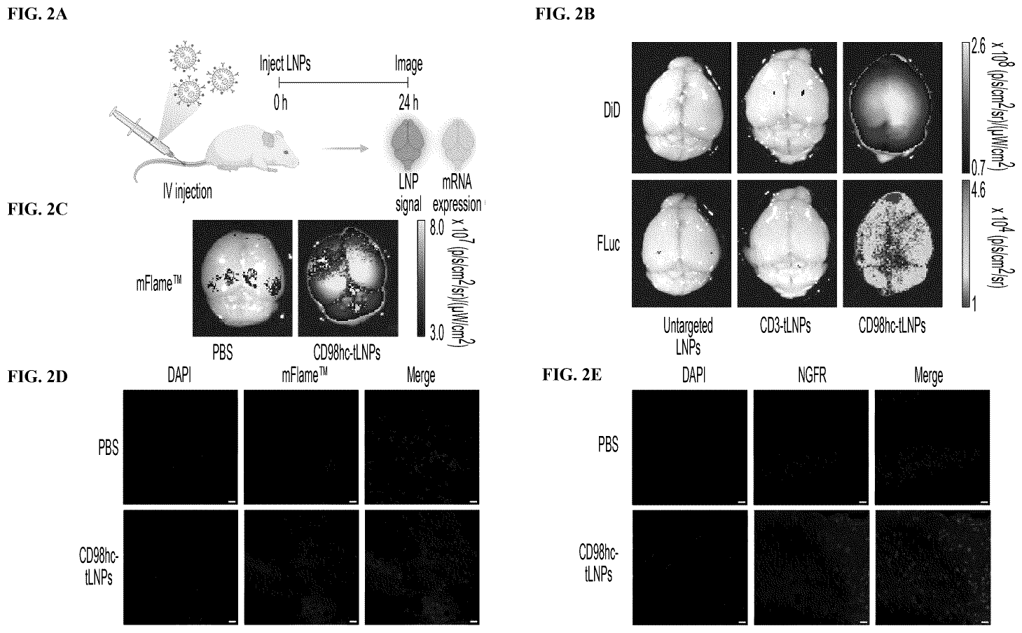

It was hypothesized that LNPs functionalized with monoclonal anti-CD98hc antibodies could deliver mRNA into the brain via receptor-mediated transcytosis after intravascular administration FIG. 1). To investigate this hypothesis, an engineered firefly luciferase (Fluc) mRNA construct was generated to impart increased mRNA stability and translatability, and packaged into CD98hc-targeted LNPs. The base LNP composition included an ionizable cationic lipid, phospholipid, sterol, and poly(ethylene glycol) (PEG) lipid. After post-insertion of a separate maleimide end group-modified PEG-lipid into the LNPs, monoclonal anti-CD98hc antibodies were coupled to the LNP surface via thiol-maleimide chemistry. A trace amount of the small, bright lipophilic dye DiD was also incorporated into LNP formulations to track in vivo biodistribution. As a first proof-of-concept experiment, CD98hc-targeted Fluc mRNA-LNPs were intravenously injected into BALB/C mice and measured luciferase expression at 24 h post-administration via ex vivo whole organ fluorescence imaging (FIG. 2A). Consistent with the hypothesis, distinct luciferase activity and a strong DiD signal was observed in the brains of mice administered CD98hc-targeted mRNA-LNPs, whereas mice injected with non-functionalized Fluc mRNA-LNPs and control anti-CD3 antibody-targeted Fluc mRNA-LNPs failed to produce any luciferase expression or DiD signal in the brain (FIG. 2B). To determine whether this phenomenon was reproducible across other types of mRNAs, the inventor also produced separate mRNA-LNP formulations with mFlame™ mRNA (an extremely bright and stable fluorescent mRNA detectable via ex vivo whole organ fluorescence imaging) and truncated nerve growth factor receptor (NGFR) mRNA. Similar to earlier experiments with Fluc mRNA, only CD98hc-targeted mRNA-LNP formulations generated mFlame™ and NGFR expression in the brain 24 h post-administration as measured via whole organ fluorescence imaging and confocal microscopy, whereas control formulations failed to produce any detectable expression (FIG. 2C-e).

CD98hc-Targeted LNPs Detarget the Liver and Efficiently Deliver mRNA to the Spleen In Vivo

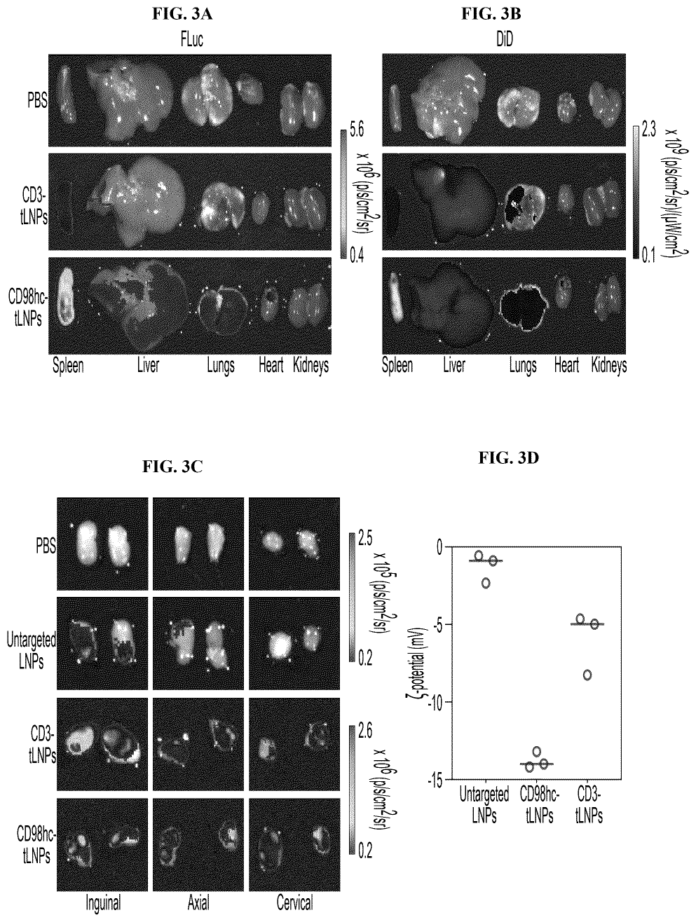

Due to first-pass hepatic metabolism, standard four-component mRNA-LNPs predominantly accumulate in the liver after intravenous administration, resulting in a liver dominant mRNA expression profile (Sabnis, et al., Molecular Therapy 26(6): 1509-1519 (2018)). However, since CD3 is an essential T cell co-receptor, systemic administration of control CD3-targeted mRNA-LNP formulations produced an mRNA expression profile largely concentrated to the spleen and lymph nodes, with reduced expression in the liver. Surprisingly, CD98hc-targeted mRNA-LNPs also displayed a peripheral mRNA biodistribution profile skewed towards secondary lymphoid tissues 24 h after intravenous injection (FIGS. 3A-B). Splenic mRNA expression was consistently higher for CD98hc-targeted mRNA-LNPs than CD3-targeted mRNA-LNPs, while similar expression profiles were observed for both formulations across different lymph nodes, including inguinal, axial and cervical nodes (FIG. 3C). By contrast, untargeted mRNA-LNPs only generated low-level mRNA expression in the spleen and inguinal lymph nodes, with no detectable mRNA expression observed in other lymph nodes. Hepatic mRNA expression was also observed for CD98hc-targeted mRNA-LNPs, although to a much lesser extent than the spleen. The strong negative net charge of the CD98hc-targeted formulations is likely a contributor to splenic and lymph node uptake (FIG. 3D). However, CD98hc is also expressed in proliferating lymphocytes (Cantor, J.; et al., The Journal of Immunology 187(2): 851-860 (2011)) and macrophages (Tsumura, et al., Cellular Immunology, 276 (1), 128-134 (2012)), suggesting that the localization of CD98hc-targeted mRNA-LNPs in secondary lymphoid tissues is also a function of epitope-specific immune cell targeting.

Localization of CD98hc-Targeted mRNA-LNPs in the Brain is a Specific Consequence of CD98hc Binding

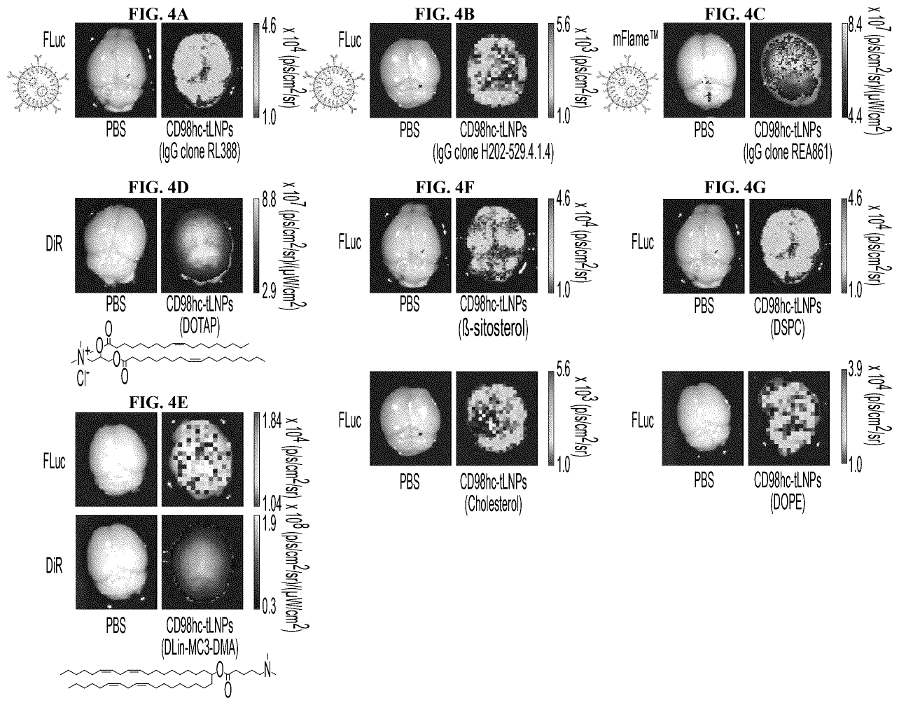

Next, the inventor sought to confirm that the CNS tropism of CD98hc-targeted mRNA-LNPs was indeed due to CD98hc binding and receptor mediated transcytosis, and not simply a consequence of non-specific interactions associated with the particular IgG antibody clone used in our initial experiments. Thus, fresh mRNA-LNPs were prepared and functionalized with a completely independent IgG antibody clone also targeted against CD98hc. Consistent with earlier results, these second generation CD98hc-targeted mRNA-LNPs also generated detectable Fluc mRNA expression and DiD signal in the mouse brain 24 h after intravenous administration LNPs (FIGS. 4A-B), as well as a similar splenic and lymph node dominant peripheral biodistribution profile (data not shown). Subsequently, another batch of mRNA-LNPs was prepared using mFlame™ mRNA instead of Fluc mRNA, and these LNPs were functionalized with a third unique anti-CD98hc IgG antibody clone, albeit using a different coupling method. Consistent with both prior CD98hc-targeted formulations, distinct mFlame™ expression was observed in the brain 24 h after intravenous administration of targeted mRNA-LNPs, whereas control mRNA-LNPs failed to produce any detectable expression in the brain (FIG. 4c).

mRNA Delivery Properties of CD98hc-Targeted LNPs are Dependent on General Lipid Classes but not Specific Lipid Structures

Having confirmed that CNS localization of CD98hc-targeted LNPs is a specific property of CD98hc binding, the inventor next sought to elucidate whether the observed mRNA delivery properties are dependent upon specific lipid structures. To this end, the lipid composition of the formulation was varied while keeping the overall molar ratios (50/10/38.5/1.5; ionizable lipid/phospholipid/sterol/PEG lipid) and antibody parameters fixed, and the resultant mRNA and LNP biodistribution profiles were measured in vivo. First, the choice of ionizable cationic lipid was varied. After demonstrating brain-specific mRNA expression in initial experiments using CD98hc-targeted LNP formulations incorporating SM-102 (the ionizable lipid used in Moderna's COVID-19 vaccine), separate LNPs were subsequently formulated with another ionizable lipid, DLin-MC3-DMA (MC3), which is used in the FDA-approved siRNA therapy Onpattro (Patisiran). The remaining lipid components (1,2-distearoyl-sn-glycero-3-phosphocholine (DSPC), β-sitosterol and 1,2-dimyristoyl-rac-glycerol-methoxy(poly(ethylene glycol)) (DMG-PEG)) were kept fixed. At 24 h post-administration, the CD98hc-targeted MC3 LNP variant produced an LNP biodistribution and mRNA expression profile consistent with the earlier formulation incorporating SM-102 (FIG. 4E). These results prove that the ability of CD98hc-targeted LNPs to target the brain does not depend upon any specific ionizable lipid structure. Next, the ionizable cationic lipid was swapped out for a permanently cationic lipid, selecting the quaternary amino lipid 1,2-dioleoyl-3-trimethylammonium-propane (DOTAP) (FIG. 4D). These CD98hc-targeted DOTAP formulated LNPs also produced detectable LNP signal in the brain and spleen 24 h after intravenous injection, in contrast to untargeted DOTAP formulated LNPs which demonstrated no brain signal and minimal splenic signal. Unlike previous targeted formulations, however, LNP biodistribution for CD98hc-targeted DOTAP formulated LNPs was also observed in the lungs (data not shown).

Next, the choice of sterol was varied while keeping all other lipid components uniform; separate LNP formulations were produced incorporating either cholesterol or β-sitosterol. In the experiments, CD98hc-targeted LNPs prepared with cholesterol and β-sitosterol both produced similar biodistribution profiles at 24 h post-administration (FIG. 4F); mRNA expression was detected in the brain while peripheral mRNA expression was skewed towards the spleen (data not shown). These results suggest that the specific choice of sterol does not play a major role in determining the pharmacokinetics of CD98hc-targeted LNPs.

It was subsequently investigated whether CD98hc-targeted LNPs formulated with different phospholipids produced distinctive mRNA and LNP biodistribution profiles. To this end, a CD98hc-targeted LNP was formulated incorporating 1,2-dioleoyl-sn-glycero-3-phosphoethanolamine (DOPE) instead of DSPC. 24 h after intravenous injection, it was observed that formulations incorporating DOPE produced a comparable in vivo mRNA expression profile to formulations incorporating DSPC (FIG. 4G). CNS and splenic mRNA expression were observed for both formulations, with no detectable difference between biodistribution profiles (data not shown).

The effect of different PEG lipid chemical structures on in vivo mRNA and LNP biodistribution was not investigated. Since the purpose of the PEG lipid is to confer colloidal stability and stealth properties to mRNA-LNP formulations, it was hypothesized that varying the PEG lipid structure would be unlikely to alter the in vivo biodistribution of CD98hc-targeted mRNA-LNPs in any meaningful way.

Collectively, these results suggest that for CD98hc-targeted LNP formulations, effective mRNA delivery to target tissues is dependent upon the presence of specific classes of lipids, rather than the exact chemical structure of each lipid.

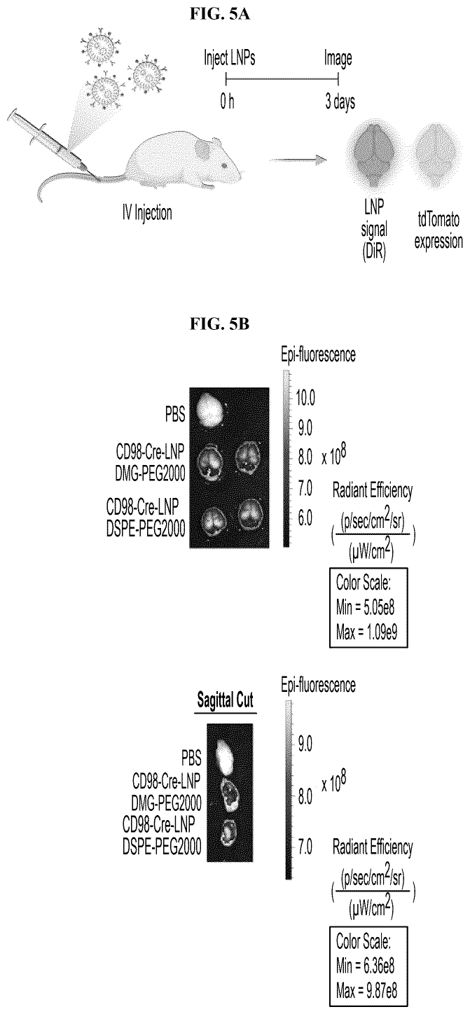

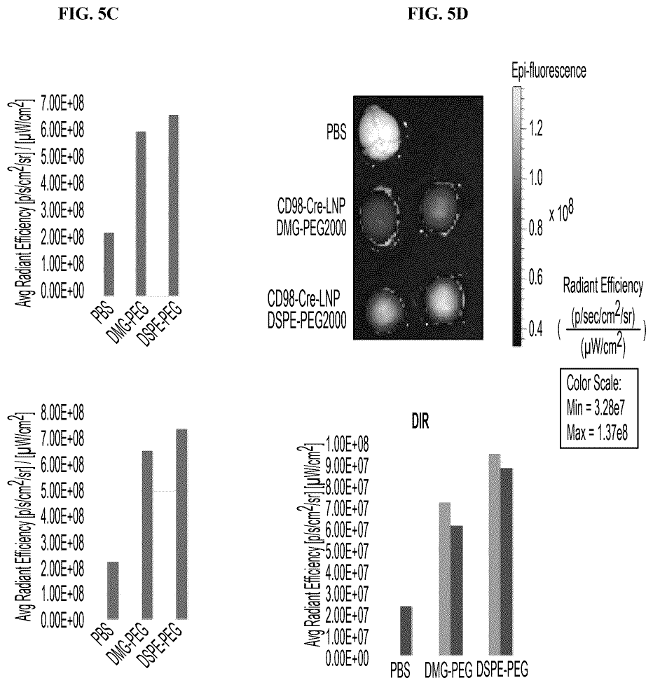

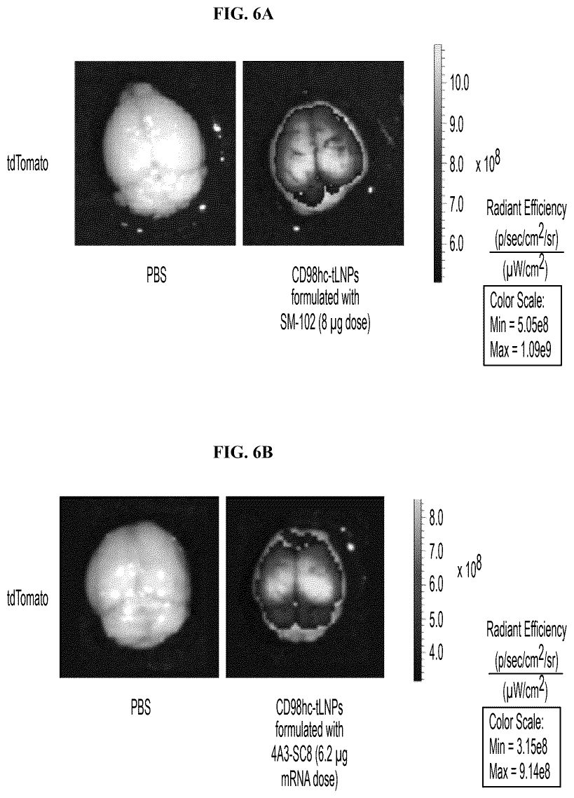

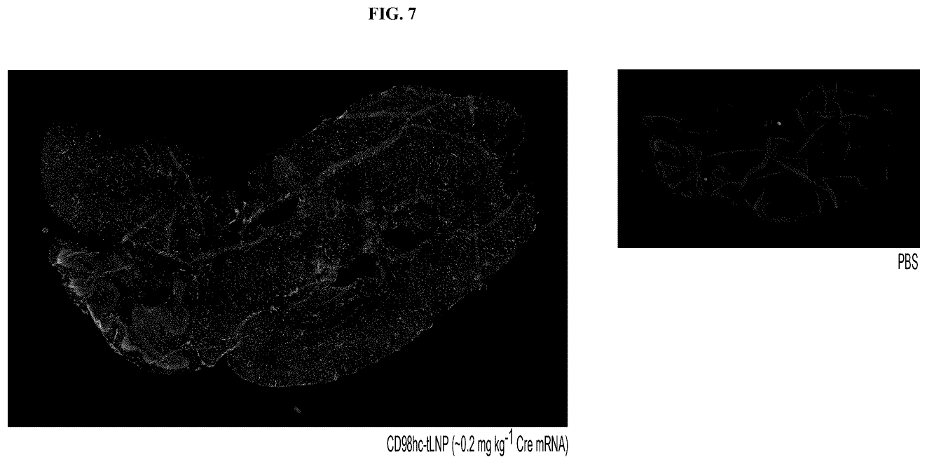

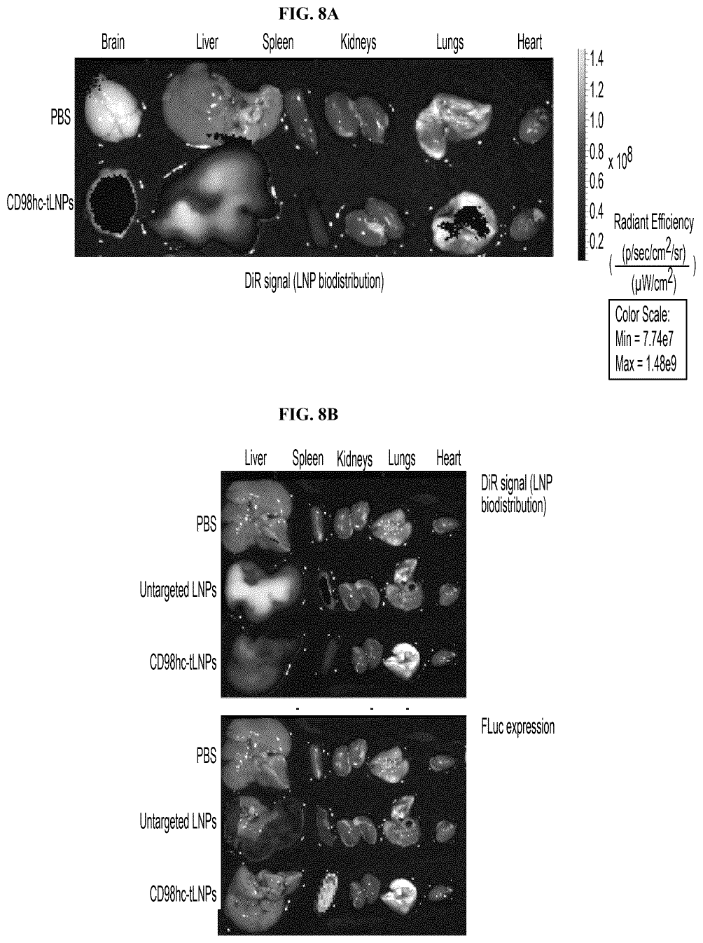

Moreover, additional experiments were also conducted with CD98hc-targeted LNPs (CD98hc-tLNPs) formulated with DSPE-PEG2000 instead of DMG-PEG2000 (FIG. 5A). This approach was also efficacious and enabled more efficient delivery of Cre mRNA to the Ai14 mouse brain after IV injection (FIGS. 5B-D). Specifically, CD98hc-tLNPs formulated with DSPE-PEG2000 incur slower PEG-lipid shedding due to the longer lipid tail length and thus stronger membrane anchoring of DSPE-PEG2000 compared to DMG-PEG2000, thus produced higher levels of whole brain tdTomato expression and whole brain DiR LNP signal in Ai14 mice 3 days after IV administration compared to CD98hc-tLNPs formulated with DMG-PEG2000. As shown in FIGS. 6A-B, the inventor has also shown that CD98hc-tLNPs are compatible with different ionizable lipids, and retain efficient brain-targeting independent of specific ionizable lipid chemistries. In particular, tdTomato expression was analysed in Ai14 mouse brains 3 days after IV injection of CD98hc-tLNPs formulated with Cre mRNA. Importantly, IV delivery of these CD98hc-tLNPs resulted in brain-wide blood brain-barrier engagement and diffuse tdTomato expression in the brain (FIG. 7), demonstrating the utility of the proposed LNPs. Lastly, analysis of the expression of the mRNA throughout the body was examined by measuring DiR signal (LNP distribution) and FLuc expression. As shown in FIGS. 8A-B, about 7% of IV injected CD98hc-tLNPs reached the brain, with significantly reduced mRNA expression in the liver compared to untargeted LNPs.

It will be understood that the invention disclosed and defined in this specification extends to all alternative combinations of two or more of the individual features mentioned or evident from the text or drawings. All of these different combinations constitute various alternative aspects of the invention.

1-46. (canceled)

47. A lipid nanoparticle (LNP) comprising a nucleic acid, wherein the LNP is conjugated to an antibody capable of transporting the LNP across the blood-brain barrier, wherein the antibody is an antibody against CD98 heavy chain (CD98hc), or a fragment thereof.

48. A pharmaceutical composition comprising a lipid nanoparticle (LNP) and a nucleic acid, wherein the LNP is conjugated to an antibody capable of transporting the LNP across the blood-brain barrier, wherein the antibody is an antibody against CD98 heavy chain (CD98hc) or a fragment thereof, preferably wherein the pharmaceutical composition comprises a pharmaceutically acceptable carrier, diluent or excipient.

49. The LNP of claim 47, wherein the LNP encapsulates the nucleic acid.

50. The LNP of claim 47, wherein the LNP comprises an ionizable lipid, a helper lipid, a sterol, and PEG-lipid or a surfactant, optionally wherein the molar ratio of ionizable lipid, helper lipid, sterol, and PEG-lipid is 50:10:38.5:1.5.

51. The LNP of claim 50, wherein the ionizable lipid is selected from the group consisting of 9-Heptadecanyl 8-{(2-hydroxyethyl)[6-oxo-6-(undecyloxy)hexyl]amino}octanoate (SM-102), DLin-MC3-DMA (MC3), 1,2-distearyloxy-N,N-dimethyl-3-aminopropane (DSDMA), 1,2-dioleyloxy-N,Ndimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA), 2,5-bis((9z,12z)-octadeca-9,12,dien-1-yloxyl)benzyl-4-(dimethylamino)butnoate (LKY750), 4-hydroxybutyl)azanediyl)bis(hexane-6,1-diyl)bis(2-hexyldecanoate (ALC0315), C12-200, 306-012B, 4A3-SC8, cKK-E12, 2,2-dilinoleyl-4-dimethylaminoethyl-[1,3]-dioxolane (DLin-KC2-DMA), and di((Z)-non-2-en-1-yl) 9-((4-(dimethylamino)butanoyl)oxy)heptadecanedioate (L319).

52. The LNP of claim 50, wherein the helper lipid is a cationic lipid selected from the group consisting of 1,2-dioleoyl-3-trimethylammonium-propane (DOTAP), 1,2-dioleoyl-s-glycero-3-ethylphosphocholine (EPC), dimethyldioctadecylammonium bromide (DDAB), and 1,2-di-O-octadecenyl-3-trimethylammonium-propane (DOTMA).

53. The LNP of claim 50, wherein the helper lipid is a phospholipid selected from the group consisting of 1,2-distearoyl-sn-glycero-3-phosphocholine (DSPC), 1,2-dioleoyl-3-trimethylammonium-propane (DOTAP), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OchemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine (DSPE), 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin and combinations thereof.

54. The LNP of claim 50, wherein the sterol is selected from the group consisting of β-sitosterol, cholesterol, 24(S)-hydroxycholesterol, 20α-hydroxycholesterol, cholesterol oleate, other cholesterol esters, fecosterol, campesterol, stigmasterol, brassicasterol, ergosterol, tomatidine, tomatine, ursolic acid and alpha-tocopherol and combinations thereof.

55. The LNP of claim 50, wherein the PEG-lipid is selected from the group consisting of PEG2000-c-DMG, PEG2000-DMG, PEG2000-DLPE, PEG2000-DMPE, PEG2000-DPPC, a PEG2000-DSPE lipid and combinations thereof.

56. The LNP of claim 50, wherein the surfactant is selected from the group consisting of a non-ionic surfactant, an anionic surfactant, a cationic surfactant or an amphoteric surfactant, preferably Polysorbate 20 or Polysorbate 80.

57. The LNP of claim 47, further comprising one or more additional helper lipids, optionally a cationic lipid, an anionic lipid, ceramide, sphingosine, sphingomyelin, cerebroside, LPC oleate, alpha-tocopherol, folate-conjugated lipid, dehydroascorbic acid-conjugated lipid or 6-O-glucose-conjugated lipid.

58. The LNP of claim 47, further comprising a maleimide end-group modified PEG-lipid, preferably a maleimide end-group modified DSPE-PEG2000 lipid.

59. The LNP of claim 47, wherein the antibody is a human CD98hc antibody.

60. The LNP of claim 59, wherein the CD98hc antibody targets human CD98hc having an amino acid sequence according to Uniprot IDs: P08195-1, P08195-2, P08195-3, P08195-4, and P08195-5.

61. The LNP of claim 59, wherein the CD98hc target is glycosylated or phosphorylated.

62. The LNP of claim 47, wherein the antibody is an IgG antibody.

63. The LNP of claim 47, wherein the antibody is a fragment selected from the group consisting of Fab, Fab′, Fab′-SH, F(ab′)2, Fv, single-domain (sdAb) and scFv.

64. The LNP of claim 47, wherein the nucleic acid is a messenger RNA (mRNA) encoding a therapeutic or diagnostic polypeptide, optionally wherein the mRNA encoding a therapeutic or diagnostic polypeptide comprises a cap, 5′ UTR, coding sequence, a 3′ UTR and a poly A tail.

65. The LNP of claim 64, wherein the mRNA comprises an optimised codon and/or a chemical modification, optionally wherein the chemical modification is modification of uridine-5′-triphosphate to 5-methoxyuridine-5′-triphosphate (5moUTP) or N1-methylpseudouridine-5′-triphosphate (m¹ψTP).

66. A method of treating or preventing a neurological disease or disorder in a subject in need thereof, comprising administering the pharmaceutical composition of claim 48 to the subject, thereby treating or preventing the neurological disease or disorder in the subject.
